(19) United States
(12) Patent Application Publication
(10) Pub. No.: US 2023/0250383 A1
(43) Pub. Date: Aug. 10, 2023

(54) Fractals in Tissue Engineering

(71) Applicants: Universiteit Twente, Enschede (NL); Ente Ospedaliero Specializzato in Gastroenterologia "Saverio De Bellis" - IRCCS, Castellana Grotte (IT)

(72) Inventors: Arturo Susarrey Arce, Enschede (NL); Silke Inge Krol, Castellana Grotte (IT); Johan Willem Berenschot, Enschede (NL); Niels Roelof Tas, Enschede (NL)

(21) Appl. No.: 18/003,195

(22) PCT Filed: Jun. 30, 2021
(86) PCT No.: PCT/NL2021/050409

§ 371 Date: Dec. 23, 2022

(30) Foreign Application Priority Data
Jun. 30, 2020 (NL) 2025961

(51) Int. Cl. C12M 1/12; C12N 5/00; C12M 3/00

(52) U.S. Cl. CPC C12M 25/14; C12N 5/0062; C12N 5/0068; C12M 21/08; C12N 2513/00

(57) ABSTRACT

The disclosure relates to a method for producing three-dimensional cell cluster on an inorganic cell culture platform comprising three-dimensional structures, preferably fractal structures. Such three-dimensional structures are useful for culturing cells and tissues, preferably in three dimensions. Such three-dimensional structures are useful for inducing differentiation, preferably of non-embryonic stem cells. In particular, such three-dimensional (3D) structures are useful for culturing primary tissue cells.

### FIELD OF THE INVENTION

The disclosure relates to a method for producing three-dimensional cell cluster on an inorganic cell culture platform comprising three-dimensional structures, preferably fractal structures. Such three-dimensional structures are useful for culturing cells and tissues, preferably in three dimensions. Such three-dimensional structures are useful for inducing differentiation, preferably of non-embryonic stem cells. In particular, such three-dimensional (3D) structures are useful for culturing primary tissue cells.

### BACKGROUND OF THE INVENTION

Studies of biology, drug discovery, diseases, and physiology are often performed in cell culture by studying cells or cell systems. Cell culture in vitro is one of the milestones for our understanding of biology in health and disease. In vitro cell culture provides an accessible and controlled environment to study cells and perform experiments.

In the past decades, various cell culture techniques and cell culture templates have been developed. The majority of experiments in biology and medicine are performed in 2D cell culture. However, 3D cell culture (spheroids) and organoid growth would be a better mimic the cell interaction and behaviour in the body. Therewith, in vitro 3D experiments could partially replace in vivo experiments. Another important field for 3D cell culture is tissue engineering, which aims at “creating functional 3D tissues using cells combined with scaffolds or devices that facilitate cell growth, organization, and differentiation.”

The growth of cells in 3D as a multicellular organoid complex, preferentially as co-culture of different cell types, is still in its infancy as it usually requires specific surface modifications or culture conditions. In order to force two dimensional 2D cell culture into the third dimension, prevention of attachment in liquid cell culture (floating spheroids) or the introduction of cells in a gel matrix is required. Floating spheroids are achieved by increasing the hydrophobicity of the culture dish surface or prevention of attachment in general (e.g., hanging drop culture, continuous stirring of the cell suspension, or by cell-repellent polymer deposition). In some templates, nanostructuring is used as “coating” aimed to induce a pattern that prevents cell attachment. In hydrogels (e.g., Matrigel, alginate, collagen) cells are seeded into the dense material to form 3D spheroids.

US 2002/182241 describes the preparation of three-dimensional templates or scaffolds that mimic blood vessels and serve as template for cell adhesion and growth. In example 1 of US 2002/182241, the preparation of scaffolds from silicon or Pyrex wafers is described, whereby channels are formed by aniotropic etching of the silicon wafers after a layer of silicon dioxde is deposited on the silicon wafer. After etching, the silicon dioxide is removed and cells are seeded and grown directly on the etched silicon or Pyrex.

These complex coating and culture techniques, along with other drawbacks (extended growth time, limited accessibility, or a low number of spheroids), somehow limit the standardized use of 3D cell culture despite their usefulness, especially in terms of predictiveness for medical applications. Therefore, there is a need for cell culture templates that allow cells to grow in the third dimension that can be used without prior surface treatment for better mimicking of the natural conditions of cells in vivo.

### SUMMARY OF THE INVENTION

The disclosure provides the following preferred embodiments.

The disclosure provides a method of producing a cell culture template with at least one three-dimensional structure having a surface maintaining a cell culture, the at least one three-dimensional structure preferably being a fractal structure, preferably produced by means of micro- and nanofabrication, the method comprising the following steps:

step 1: providing a monocrystalline substrate, preferably a monocrystalline silicon substrate;
step 2: subtracting at least one geometrical feature from the monocrystalline substrate to produce a geometrical cavity, preferably forming one or more apices, preferably an octahedral cavity or part of an octahedral cavity, in the monocrystalline substrate that renders as the initiation for a three-dimensional structure;
step 3: the growth and/or deposition of the base three-dimensional structure material, preferably a silicon oxide, preferably amorphous silicon dioxide, on the surface of the geometrical features in the substrate to form the three-dimensional structure;
step 4: bonding of the at least one three-dimensional structure to a surface of a support base, preferably borosilicate glass, in particular whereby the support base is bonded to the at least one three-dimensional structure at the surface on which the base three-dimensional structure material is grown or deposited; and
step 5: removal of the bulk-monocrystalline substrate around the at least one three-dimensional structure;
wherein after removal of the bulk-monocrystalline substrate the surface of the at least one three-dimensional structure is provided with cells under growth permitting conditions to produce the cell culture template, in particular whereby said cells are provided to the at least one three-dimensional structure at the surface comprising the base three-dimensional material.

Preferably, the method further comprises the following steps:

step 6: treating the monocrystalline substrate to form a protective layer which is compatible with the next steps;
step 7: create one or more apertures in the protective layer, preferably an aperture at each of the one or more apices, which is compatible with the following steps;
step 8: subtracting at least one geometrical feature, preferably an octahedron or part of an octahedron, in the monocrystalline substrate through the one or more apertures; followed by stripping the protective layer;
wherein steps 6-8 are performed between step 2 and step 3 of the method of claim 1, optionally repeating steps 6-8 one or more times to create the at least one three-dimensional structure with a higher level of complexity,

preferably wherein steps 6-8 of the method are repeated 2-10 times, preferably 2-5 times to produce three-dimensional structures with higher complexity.

The protective layer is preferably the base three-dimensional structure material as described herein, preferably silicon oxide or silicon nitride, more preferably silicon dioxide.

Preferably, the method for producing a cell culture template comprising at least one three-dimensional structure as described herein, wherein the cavity formed in the monocrystalline substrate of step 2 is accessible from outside the substrate through an opening provided in the substrate by a pre-subtracting directional step, preferably the opening in the substrate having a relatively large width compared to an average width of the cavity, more preferably, the opening forming a widest part of the cavity formed in the substrate.

Preferably, the method for producing a cell culture template comprising at least one three-dimensional structure as described herein, wherein the subtracting is performed by means of anisotropic etching.

Preferably, the method for producing a cell culture template comprising at least one three-dimensional structure as described herein, wherein the provided monocrystalline substrate is silicon, whereby thermal oxidation results in a layer of silicon oxide, preferably amorphous silicon dioxide, whereby in step 3 a layer of silicon dioxide is deposited and whereby in step 5 the bulk-silicon around the formed three-dimensional structure is removed.

Preferably, the method for producing a cell culture template comprising at least one three-dimensional structure as described herein, whereby step 7 is left out at the last round of preparation to produce three-dimensional structures having closed apices.

Preferably, the method for producing a cell culture template comprising at least one three-dimensional structure as described herein, wherein

the three-dimensional structure comprises a surface defining a regular pattern of protrusions; the protrusions are built up from octahedral structures; and the octahedral structures are becoming narrower to the outside of the three-dimensional structure.

Preferably, the method for producing a cell culture template comprising at least one three-dimensional structure as described herein, wherein the three-dimensional structure has any of the following topographies:
a pyramid (G0),
a pyramid with on the apex an octahedral (G1),
a pyramid with on the apex an octahedral and on each apex of the octahedral a second level of octahedral structures (G2),
a pyramid with on the apex an octahedral and on each apex of the octahedral a second level of octahedral structures and on each apex of the second level a third level of octahedral structures (G3), or
a pyramid with on the apex an octahedral and on each apex of the octahedral a second level of octahedral structures and on each apex of the second level a third level of octahedral structures and on each apex of the third level a fourth level of octahedral structures (G4),
a pyramid with on the apex an octahedral and on each apex of the octahedral a second level of octahedral structures and on each apex of the second level a third level of octahedral structures and on each apex of the third level a fourth level of octahedral structures (G4), on each apex of the n−1th level a nth level of octahedral structures (Gn) n being 5-10.

Preferably, the method for producing a cell culture template comprising at least one three-dimensional structure as described herein, whereby the three-dimensional structure is sterilized before growing cells, preferably the three-dimensional structure is sterilized by any one of UV, chemical means and high temperature treatment.

Preferably, the method for producing a cell culture template comprising at least one three-dimensional structure as described herein, wherein the at least one three-dimensional structure comprises multiple three-dimensional structures and wherein the multiple three-dimensional structures are placed on the surface of the support base in a lattice configuration, preferably a square or hexagonal lattice configuration.

Preferably, the method for producing a cell culture template as described herein, wherein the bulk-monocrystalline substrate is partially etched away with remaining substrate at least partially covering at least one of the multiple three-dimensional structures.

Preferably, the method for producing a cell culture template as described herein, wherein the bulk monocrystalline substrate is partially etched away to create multiple compartments with one or more three-dimensional structures exposed.

Preferably, the method for producing a cell culture template comprising at least one three-dimensional structure as described herein, wherein the cells are in the form of a tissue or organoid.

Preferably, the method for producing a cell culture template comprising at least one three-dimensional structure as described herein, wherein the cell culture template further comprises at least one insulator, preferably the insulator is a three-dimensional structure of amorphous silicon dioxide.

Preferably, the method for producing a cell culture template comprising at least one three-dimensional structure as described herein, wherein the cell culture template further comprises at least one metal portion, preferably the metal portion is embedded or patterned within the three-dimensional structure.

Preferably, the method for producing a cell culture template comprising at least one three-dimensional structure as described herein, wherein the three-dimensional structures are used for external stimulation of the culture.

Preferably, the method for producing a cell culture template comprising at least one three-dimensional structure as described herein, wherein electrodes are used for cell stimulation, preferably wherein at least part of the three-dimensional structures function as electrodes.

Preferably, the method for producing a cell culture template comprising at least one three-dimensional structure as described herein, wherein the apices are open and the solutions can be supplied through these apices in the cells culture.

The disclosure provides a cell culture template for growing and maintaining a cell culture, in particular a cell culture comprising primary cells, the cell culture template comprising cells seeded on a cell growth surface, for example a surface of an amorphous silicon dioxide, the surface defined by at least one three-dimensional fractal structure carried on a support base, for example a layer of borosilicate glass.

Preferably, the cell culture template as described herein, wherein the surface is defined by a multitude of, preferably at least almost identical, three-dimensional fractal structures evenly distributed on the support layer.

Preferably, the cell culture template as described herein, wherein some of the three-dimensional fractal structures of the multitude of three-dimensional fractal structures on the support layer are covered by monocrystalline substrate with the other three-dimensional fractal structures of the multitude of three-dimensional fractal structures being exposed, i.e. free of monocrystalline, to form the cell growth surface.

Preferably, the cell culture template as described herein, wherein the monocrystalline substrate is arranged to define one or more cell growth compartments having one or more exposed fractals.

Preferably, the cell culture template as described herein, wherein a lid is provided on a side of the cell layer opposite of the cell growth surface on top of and supported by the monocrystalline substrate.

The disclosure provides a method for culturing cells, comprising providing a cell culture template obtainable by a method according to the invention, and culturing the cells.

Preferably, the method for culturing cells or tissues as described herein, wherein the cells are primary cells, preferably primary tumour cells.

Preferably, the method for culturing cells or tissues as described herein, wherein the cells are primary cells, preferably primary tissue cells.

Preferably, the method for culturing cells or tissues as described herein, wherein the cells are cancer-associated fibroblasts (CAFs).

Preferably, the method for culturing cells or tissues as described herein, wherein the cells are cancer-associated fibroblasts (CAFs) activated by the material, shape, and/or the pattern of the three-dimensional structures.

Preferably, the method for culturing cells or tissues as described herein, wherein the cells are stem cells, preferably mesenchymal stem cells, adult stem cells, adipose adult stem cells and/or induced pluripotent stem cells.

Preferably, the method for culturing cells or tissues as described herein, wherein the cells form a multicellular organoid or tissue.

Preferably, the method for culturing cells or tissues as described herein, wherein the cells undergo stem cell differentiation initiated by the pyramidal shape and the distance of the three-dimensional structures.

Preferably, the method for culturing cells or tissues as described herein, wherein the cells are grown and be preserved in non-optimal growth conditions.

The disclosure further provides a cell culture template comprising at least one three-dimensional structure obtainable by a method as described herein, composed of amorphous silicon dioxide and cells attached to the structure. Preferably, the three-dimensional structure of amorphous silicon dioxide consists of SiO₂.

The disclosure further provides a method for producing a three-dimensional structure for cell culture, preferably the three-dimensional structure is a fractal structure, produced by means of micro- and nanofabrication comprising the following steps:

step 1: providing a monocrystalline substrate, preferably a monocrystalline silicon substrate;
step 2: subtracting at least one geometrical feature from the monocrystalline substrate to produce a geometrical cavity, preferably forming one or more apices, preferably an octahedral cavity or part of an octahedral cavity, in the monocrystalline substrate that renders as the initiation for a three-dimensional structure;
step 3: the growth and/or deposition of the base three-dimensional structure material, preferably a silicon oxide, preferably amorphous silicon dioxide, on the surface of the geometrical features in the substrate to form the three-dimensional structure;
step 4: bonding of the at least one three-dimensional structure to a surface of a support base, preferably borosilicate glass; and
step 5: removal of the bulk-monocrystalline substrate around the at least one three-dimensional structure;
wherein after removal of the bulk-monocrystalline substrate the surface of the at least one three-dimensional structure is provided with cells under growth permitting conditions to produce the cell culture template,
optionally, wherein the method further comprises the following steps:
step 6: treating the monocrystalline substrate to form a protective layer which is compatible with the next steps;
step 7: create one or more apertures in the protective layer, preferably an aperture at each of the one or more apices, which is compatible with the following steps;
step 8: subtracting at least one geometrical feature, preferably an octahedron or part of an octahedron, in the monocrystalline substrate through the one or more apertures; followed by stripping the protective layer;
wherein steps 6-8 are performed between step 2 and step 3, optionally repeating steps 6-8 one or more times to create the at least one three-dimensional structure with a higher level of complexity.

### BRIEF DESCRIPTION OF THE DRAWINGS

FIG. 1: Initiator: Etching of the monocrystalline substrate to subtract at least one, or part of one geometrical feature with anisotropic etching to produce a geometrical cavity. The displayed geometrical cavities are an octahedral cavity or a part of an octahedral cavity. This cavity renders as the initiation for a three-dimensional structure, thereby preferably forming one or more apices. In the middle planes, the octahedral cavity in the monocrystalline substrate has broad access to the outside of the substrate. In the right plane, the octahedral cavity in the monocrystalline substrate has the widest point of the octahedral shape as opening and access to the outside of the substrate. G1: Schematic display of the second round of anisotropic etching, creating octahedral cavities at each apex of the previous cavity in the monocrystalline substrate.

FIG. 2. Scanning electron micrographs of the amorphous silicon dioxide fractals. A) square orientation with a 20 μm pitch; B) hexagonal orientation with a 12 μm pitch; the structure of C) G0; D) G1; E) G2; F) G3; G) G4. The size bar in A) and B) indicates 20 μm; for the images in C)-G) it is 2 μm.

FIG. 3. CAFs 13 days after seeding on hexagonal oriented inorganic fractal surfaces. A) control; B) G0; C) G1; D) G2; E) G3; F) G4. The blue fluorescent signal is due to DAPI staining of the nucleus while the red fluorescence is related to the TRITC-phalloidin which labels the actin filaments of the cytoskeleton. The underlying fractals were visualized by transmission light. Arrows indicate elongated nuclei. The size bar indicates 100 μm.

FIG. 4. CAF cells 8 days after seeding on square oriented inorganic fractal surfaces. The nuclei are stained by DAPI (blue) and the actin filaments by TRITC-phalloidin (red). The size bar in the fluorescence micrographs indicates 100 μm and in the EM images 20 μm.

FIG. 5. Magnified view on CAFs grown for 8 days on G3 square configuration. The nuclei are stained with DAPI (blue) and the actin filaments with TRITC-labelled phalloidin (red). Lamellipodia are brighter red due to actin accumulation. The nuclei are elongated but located between the fractals.

FIG. 6. (A) Light microscopy of CAF cells at day 1, and (B) tumor spheroids on CAF cells after day 6 of culture on G0Sqr.

FIG. 7: Light microscopy images of hADSC grown on square configuration after 24 h (middle panel) and 48 h (lower panel). The upper panel shows the corresponding fractal structures.

FIG. 8: Human adipose-derived stem cells (hADSC) after 1 day of culture on G2Hex. The green signal indicates nestin, a biomarker for neurospheres while the red signal is representative for the presence of NeuN, a nucelar marker of mature neurons. The blue signal is due to a staining of the nuleus.

FIG. 9: (Upper panels) Light microscopy images of COLO205 on different fractal structured surfaces 48 h after seeding. The cells only form 2D cell sheets. (Lower panel) The cells also grow in sheets on a cell-repellent PEG6000 (Carlo Erba) coating.

FIG. 10: Selective opening of the thermally grown amorphous silicon dioxide at the apex of the pyramidal pit after HF etching. Note that stress-induced oxidation retardation is more pronounced in concave corners when more than two planes intersect.

FIG. 11: A. Top and middle: 3D and top view schematic representations of 2, 3 and 4 intersecting (111)-Si planes. Bottom: top view SEM-images of insections of 2, 3 and 4 (111)-Si planes upon etching in HF: time dependent opening of the apices is visible. B. Remaining oxide thickness in apices and ribbons as a function of etching time in 1% HF (starting oxide thickness 160 nm (left) or 88 nm (right)): within the time window Δt only the apices are opened. Process fabrication advancements can lead to the starting oxide thickness 25 nm.

FIG. 12: The three-dimensional structures are bonded to a glass surface. Subsequently the monocrystalline substrated may be thinned before etching the monocrystalline substrate. The monocrystalline substrate can be etched away partially, whereby part of the three-dimensional structures becomes available, for example for cell culture purposes.

FIG. 13: Analysis of epithelial, stemness, and mesenchymal markers of CAFs enriched cell populations isolated from HCC primary tumors of 3 patients (P1, P2, P3). Percentage of positive cells and/or mean fluorescence intensity of antibody-stained cell populations (MFI, expressed as arithmetic (A-Mean) and geometric (G-Mean) mean) are reported. Fluorescence values are normalized to control/isotype related signals.

FIG. 14: (A) First passage in 2D cell culture of an isolate of CAFs from primary hepatocarcinoma at the stained with antibodies for Vimentin (red) and α-SMA (green), a marker for activated fibroblasts. The nuclei were stained with DAPI (blue). (B) Cell clusters and spheroids on G0Hex formed by enriched CAFs isolated from hepatocarcinoma of 3 patients and cultured for 6 days. 100 μm.

FIG. 15: Spheroids grown on G0Hex templates. (A) Z-stack of confocal micrograph of two spheroids on a α-SMA (red) positive 2D CAF layer. The nuclei were stained with DAPI (blue). (B) Confocal image of the spheroid. Tumor cells are positive for AFP (green) enwrapped by CAFs positive for α-SMA (red) (arrow). The 2D cell layer consists of CAFs and connects the tumor with the cell layer. DAPI stains the nucleus (blue).

FIG. 16: The cells on the fractal template were stained for α-SMA (red), AFP (green), and the nucleus (DAPI, blue) (A) peritumoral tissue on G0Hex. No AFP signal due to absence of tumor cells. (B) tumor tissue on G0Sqr, and (C) tumor tissue on G1Hex. No AFP signal due to exclusive growth of CAFs. The scale bar indicates 100 μm.

FIG. 17: (A) Epifluorescence image of spheroids grown from HLF cell line on G0Hex at day 4, The inset shows only the DAPI signal and the arrow indicates exemplarily the size of spheroids considered for size distribution. (B) Diagram of the size distribution of the spheroids on fractals as determined by image analysis with ImageJ. (C) light microscopy of HLF cell spheroid embedded in Matrigel at day 13. (D) Diagram of the spheroid size distribution in Matrigel as determined by image analysis with ImageJ.

### DETAILED DESCRIPTION OF THE DISCLOSED EMBODIMENTS

The disclosure provides a method for producing three-dimensional cell cluster on an inorganic cell culture template comprising three-dimensional structures, preferably fractal structures. The cell culture template as describe herein can contribute to cell culture of primary cells and/or tissue engineering. The cell culture template can be used for various cell culture purposes, for example 3D cell culture, induce stem cell differentiation, and culturing multicellular organoids.

The disclosure provides a method of producing a cell culture template with at least one three-dimensional structure having a surface maintaining a cell culture, the at least one three-dimensional structure preferably being a fractal structure, preferably produced by means of micro- and nanofabrication, the method comprising the following steps:

step 1: providing a monocrystalline substrate, preferably a monocrystalline silicon substrate;
step 2: subtracting at least one geometrical feature from the monocrystalline substrate to produce a geometrical cavity, preferably forming one or more apices, preferably an octahedral cavity or part of an octahedral cavity, in the monocrystalline substrate that renders as the initiation for a three-dimensional structure;
step 3: the growth and/or deposition of the base three-dimensional structure material, preferably a silicon oxide, preferably amorphous silicon dioxide, on the surface of the geometrical features in the substrate to form the three-dimensional structure;
step 4: bonding of the at least one three-dimensional structure to a surface of a support base, preferably borosilicate glass; and
step 5: removal of the bulk-monocrystalline substrate around the at least one three-dimensional structure;
wherein after removal of the bulk-monocrystalline substrate the surface of the at least one three-dimensional structure is provided with cells under growth permitting conditions to produce the cell culture template.

Preferably, the method further comprises the following steps:

step 6: treating the monocrystalline substrate to form a protective layer which is compatible with the next steps;
step 7: create one or more apertures in the protective layer, preferably an aperture at each of the one or more apices, which is compatible with the following steps;
step 8: subtracting at least one geometrical feature, preferably an octahedron or part of an octahedron, in the monocrystalline substrate through the one or more apertures; followed by stripping the protective layer;
wherein steps 6-8 are performed between step 2 and step 3 of the method of claim 1, optionally repeating steps 6-8 one or more times to create the at least one three-dimensional structure with a higher level of complexity,
preferably wherein steps 6-8 of the method are repeated 2-10 times, preferably 2-5 times to produce three-dimensional structures with higher complexity.

A cell culture template is a product that can be used to culture and grow cells. In particular, the term “cell culture template” refers to the three-dimensional structure, in particular a scaffold, that is prepared with a method of the invention on which cells can be cultured and grown. A cell culture template comprises at least one template which can be used to grow the cells in a cell culture medium. The template comprises a surface to which cells can attach.

The cell culture template of the present disclosure comprises at least one three-dimensional structure. Such a three-dimensional structure can be placed on the surface of the template. The structure can rise above the surface and increase the surface area. Preferably, the structure has a maximum height of between 0.1 and 50 μm above the surface. In preferred embodiments, the structures are oriented perpendicular to the bottom surface and have a dimension in the range of 1 nm to 100 μm, preferably 50 nm to 50 μm. In preferred embodiments, the volume and area of the three-dimensional structure are defined by the size of the first geometrical cavity, preferably the areal dimensions, also called the footprint, of the first geometrical shape are between 1 and 2500 μm2.

Cells in the cell culture template may attach to the three-dimensional structures. Preferably, the three-dimensional structure is a 3D nanostructure having a nano-substructure.

In preferred embodiments, the three-dimensional structure in the cell culture template is a fractal structure. Fractal structures exhibit similar patterns at different scales called self-similarity. As used herein, the term “fractal” means and includes a pattern (i.e., shape or geometry) that can be repeatedly divided into smaller parts or repeatedly multiplied into more significant parts that are the same or similar to the original pattern (i.e., shape or geometry).

The one or more three-dimensional structure of the cell culture template is produced by micro- and nanofabrication. In microtechnology, the term “micro” means that the relevant dimension is in the micrometer range, preferably but not exclusively to less than 100 μm. In nanotechnology, the term “nano” means that the relevant dimension is less than 100 nm. In this application, the term “nano” also encompasses structures with a relevant dimension up to hundreds of microns (μm), preferably between 100 microns (μm) and 10 microns (μm). The lower limit is about 1 nm, preferably about 5 or 100 nm.

The produced three-dimensional structure has a size between 10 nm and 100 μm. In preferred embodiments, the three-dimensional structures have a size between 1 and 50 μm, more preferably between 1 and 25 μm.

The three-dimensional structure of the cell culture template is produced using a monocrystalline substrate. A single-crystal or monocrystalline solid is a material in which the crystal lattice of the entire sample is continuous and unbroken to the edges of the sample, with no grain boundaries. Monocrystalline substrates are composed of a single crystal throughout, while polycrystalline is composed of an aggregate of very small crystals in random orientations. Examples of monocrystalline are monocrystalline silicon, sapphire, Quartz, Ge (germanium), or GaN (gallium nitride).

In preferred embodiments, the monocrystalline substrate is monocrystalline silicon. Monocrystalline silicon, is also called single-crystal silicon, in short, mono c-Si or mono-Si. It consists of silicon in which the crystal lattice of the entire solid is continuous, unbroken to its edges, and free of any grain boundaries.

Silicon is tetrahedrally coordinated by oxygen in the low-pressure SiO2 polymorphs; quartz, tridymite, cristobalite, and in its high-pressure polymorph coesite. Silicon is coordinated by six oxygens in the high-pressure SiO2 polymorph stishovite.

To produce the three-dimensional structure, at least one or more geometrical feature is subtracted from the monocrystalline substrate. The geometrical feature can have various shapes, such as a pyramid, an octahedron, a tetrahedron, a cube, a cuboid, or a cone. Preferably the geometrical shape has one or more apices. In preferred embodiments, the geometrical feature has the shape of an octahedron.

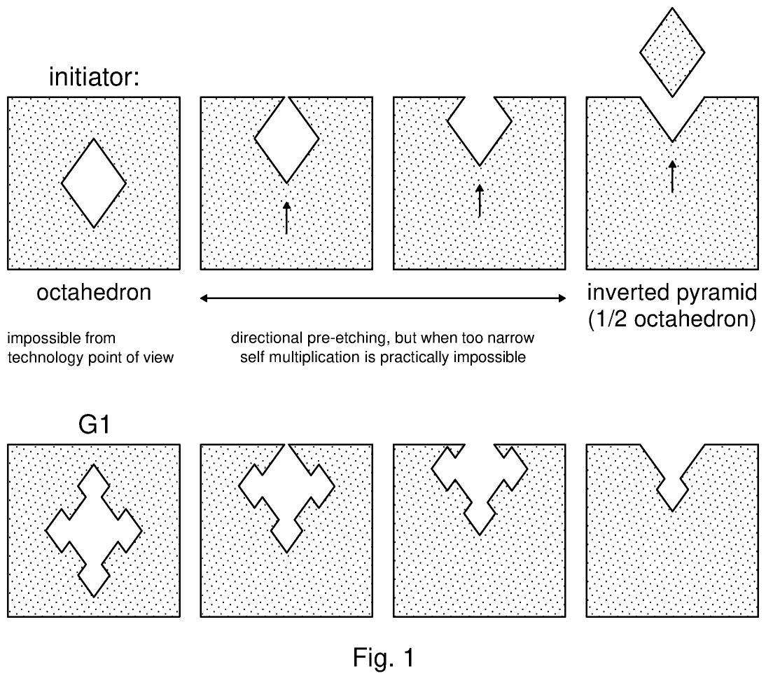

In some embodiments, the geometrical feature can be subtracted from the substrate partially. For example, three quarters, half or a quarter of the shape, can be subtracted from the monocrystalline substrate. After subtracting the geometrical shape, there is a geometrical cavity in the monocrystalline substrate. This cavity is also called the initiator cavity. FIG. 1 schematically shows an octahedron structure being subtracted partially or entirely in a monocrystalline substrate.

In preferred embodiments, the geometrical cavity is an octahedral cavity in the monocrystalline substrate that renders as the initiation for a three-dimensional structure, thereby preferably forming one or more apices as displayed in FIG. 1 (initiator).

The geometrical feature can be subtracted from the monocrystalline substrate by various methods for removal of material. For example, the geometrical feature can be subtracted by a subtraction step performed by etching or by drilling. Preferably subtraction of material from the monocrystalline substrate is performed by using etching. For example the geometrical cavity is etched in the substrate by means of anisotropic etching Anisotropic etching is a subtractive microfabrication technique that aims to remove material in specific directions to obtain a geometrical shape. Preferably, the wet etching technique can be used as anisotropic etching. Wet techniques exploit the crystalline properties of a structure to etch in directions governed by crystallographic orientation. In some embodiments, potassium hydroxide (KOH) is used for anisotropic etching of the monocrystalline substrate.

After subtracting the geometrical feature from the monocrystalline substrate, the resulting geometrical cavity in the monocrystalline substrate is treated to form a protective layer. In some embodiment, the base three-dimensional structure material as described herein, preferably silicon oxide or silicon nitride, more preferably silicon dioxide.

In some embodiments, the surface defining the cavity is formed by a layer of thermally grown oxide and a layer of silicon nitride. The layer of silicon nitride can be applied by low-pressure chemical vapor deposited (LPCVD), followed by corner lithography, and local oxidation of silicon. Next, selective stripping of remaining nitride and the underlying thin oxide is followed by anisotropic etching step of silicon.

In other embodiments, the treatment to form the protective layer is thermal oxidation. This amorphous silicon dioxide layer is conformally grown, except at the concave corners.

In some embodiments, the treatment to form a protective layer is thermal oxidation. The formed geometrical cavity is exposed to thermal oxidation at a temperature between 950-1500 degrees Celsius. At this temperature, the surfaces of the subtracted structure will oxidize. The resulting silicon oxide forms a protective layer. The thickness of the layer depends on the temperature and the duration of the thermal oxidation step. In preferred embodiments, the oxide layer is at least 25 nm thick, a preferable thickness is 160 nm. In some embodiments, the oxide layer is between 25 and 160 nm thick, in more preferred embodiments the oxide layer is between 88 and 160 nm thick.

In preferred embodiments, the monocrystalline substrate is monocrystalline silicon. Thermal oxidation of monocrystalline silicon will result in a protective layer of silicon oxide. In preferred embodiments, the thermal oxidation of silicon is performed at 1100 degrees Celsius. The oxidation of silicon results in a conformal layer of silicon dioxide, preferably amorphous over the silicon crystal. In this process, a conformal layer around convex corners is obtained. In intersections of multiple planes, e.g., three or four planes, oxide sharpening occurs. This aspect yields the possibility to solely remove the silicon oxide from apices by means of timed isotropic etching, while the oxide layer remains in ribbons and on planes. In some embodiments, a process like, plasma oxidation of silicon, anodic oxidation of silicon, or nitridation (by means of thermal conversion of silicon into nitride) can be applied to create a protective layer.

In the next step, an aperture is created at every apex in the protective layer. This aperture allows subtraction of an additional layer of cavities to create multilevel three-dimensional structures. Various techniques can be used to make an aperture, for example, corner lithography or timed isotropic etching.

In some embodiments, the apertures are created by means of timed isotropic etching. In this technique, the aperture is created by solely removing the protective layer from the apices. This can be done by timed wet etching using hydrogen fluoride, e.g., 1% hydrogen fluoride. Alternatively, for the fabrication of apertures, other methods might apply, for example, low-temperature oxidation and selective etching.

The one or more apertures are used to apply another round of subtracting at least one or part of one geometrical feature of geometrical shape in the monocrystalline substrate. In preferred embodiments, the geometrical shape is an octahedron. The subtracting is performed through the one or more apertures formed at the one or more apices. FIG. 1 (G1) schematically shows the second round of subtracting, creating octahedral cavities at each apex of the previous cavity. For example, the next round of geometrical cavities can be created by selectively etching at each apex the underlying silicon with anisotropic etching in TMAH (tetramethylammonium hydroxide). This etching step will form cavities at all apices simultaneously.

Repetition of the sequence of anisotropic etching of the monocrystalline substrate, thermal oxidation, and isotropic etching of the protection layer to create an aperture results in multilevel three-dimensional structures. In some embodiments, this sequence of steps of the production method is repeated to create a three-dimensional structure with a higher level of complexity. Each following layer of the structure will comprise smaller geometrical cavities.

After growth and/or deposition of a protective layer to the subtracted geometrical cavity, an aperture is made at each apex of the outer layer of the geometrical shapes. The aperture is used to apply another round of subtracting at least one or part of one geometrical feature of geometrical shape in the monocrystalline substrate. In preferred embodiments, the geometrical shape is an octahedron. The subtracting is performed through the one or more apertures formed at the one or more apices. After a new layer of geometrical cavities is formed the protective material is stripped from the geometrical cavities.

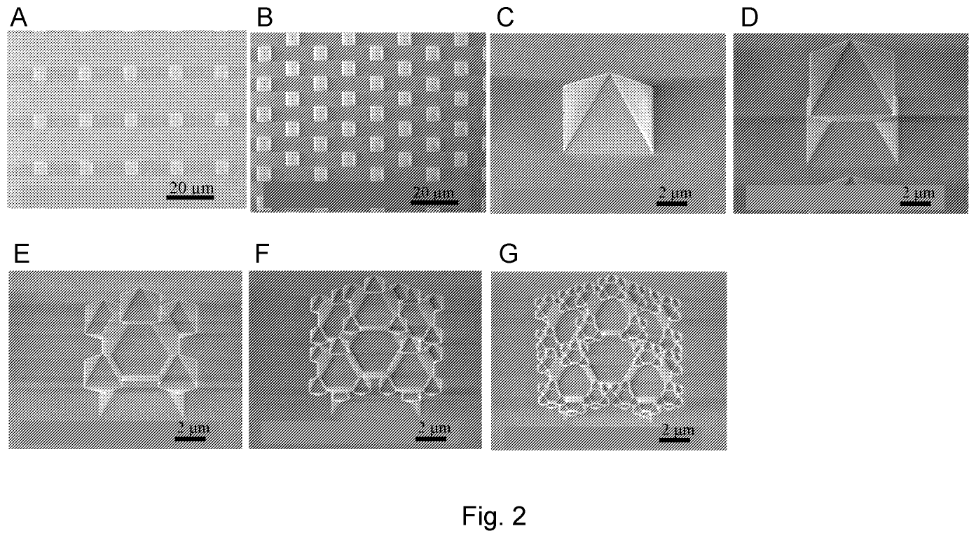

As an example, FIGS. 2a) and 2b) show the top view scanning electron micrographs (SEM) of two different layouts of the initiator, configured in a square or hexagonal lattice. FIG. 2c) shows a tilted view of a single initiator feature, as sketched in the most right image of FIG. 1. Exemplary structures on a geometrical shape of octahedrons are shown. FIG. 2C shows a simple three-dimensional structure that can be created with 1 round of subtraction. FIG. 2D shows a three-dimensional structure that can be created with 2 rounds of subtraction. FIG. 2E shows a three-dimensional structure that can be created with 3 rounds of subtraction. FIG. 2F shows a three-dimensional structure that can be created with 4 rounds of subtraction. And FIG. 2G shows a three-dimensional structure that can be created with 5 rounds of subtraction.

When the desired level of complexity is reached, a new layer is grown and/or deposited on the entire geometrical cavity. This layer can be made of various materials. For example, the layer can be grown by oxidation or nitridation. Alternatively, the layer can be created by nitride or oxide deposition. The material should be compatible with cell growth because the cells are provided to the at least one three-dimensional structure at the surface comprising this layer. After removal of the bulk-monocrystalline structure, this created layer will form the three-dimensional structure. Therefore, this layer should have a thickness sufficient to create a self-contained structure. While not wishing to be bound by theory, the material, the thickness of the material and the form of the structure together contribute to the strength of the structure. The structure should be sturdy enough to carry cells that potentially grow on the structure. In preferred embodiments, the formed layer is at least 25 nm thick, more preferably at least 50 nm thick.

In some embodiments, the silicon undergoes thermal oxidation to form a layer. The formed geometrical cavity is exposed to thermal oxidation at a temperature between 950-1500 degrees Celsius. At this temperature, the surfaces of the subtracted structure will oxidize, resulting in a layer of silicon oxide. The thickness of the layer depends on the temperature and the duration of the thermal oxidation step. In preferred embodiments, the oxide layer is at least 25 nm thick, a preferable thickness is 160 nm. In some embodiments, the oxide layer is between 25 and 160 nm thick, in more preferred embodiments the oxide layer is between 88 and 160 nm thick.

After producing the three-dimensional structure, the outside of the end-grown or deposited layer forms the functional layer of the structure and will be the outer surface. The cells will use this outer surface to attach and/or grow on. If the layer is grown, for example by thermal oxidation, the layer will grow from the surface of the cavity and will grow to the outside. Thus, the outer-layer which will become the surface of the three-dimensional structure is formed last.

Next, the produced one or more three-dimensional structures are bonded to a surface, the support base, in particular the one or more three-dimensional structures are bonded to the support base at the surface on which the base three-dimensional structure material is grown or deposited. Preferably, the surface is suitable for cell culture purposes. Suitable surfaces may be ceramics, glass, or plastic surfaces, such as:

Ceramic: silicon nitride, alumina, zirconia;

Glass: borosilicate glass, and soda-lime glass;

Polymer: polystyrene, permanox, polydimethylsiloxane;

In preferred embodiments, the one or more three dimensional structures are bonded to a surface of borosilicate glass.

The produced one or more three-dimensional structures can be bonded to a surface by various techniques. In some embodiments, the structures are bonded to the surface by electrostatic bonding. In preferred embodiments, the structures are bonded to the surface by anodic bonding. For example, anodic bonding with a Mempax glass wafer at 400° C.

Subsequently, the bulk-monocrystalline substrate around the formed three-dimensional structures is removed. The bulk-monocrystalline can be removed by a wet-etching step. For example, removal of the bulk-monocrystalline substrate, preferably silicon, is done with prolonged exposure to tetramethylammonium hydroxide. The outside of the three-dimensional structure is now accessible, for example, for cells to attach. After removal of the bulk-monocrystalline substrate, the surface of the three-dimensional structure is seeded and/or provided with cells under growth permitting conditions to produce the cell culture template. In particular the cells are provided to the at least one three-dimensional structure at the surface comprising the base three-dimensional material, in particular silcon oxide or nitride, more in particular silicon dioxide or nitride.

In vitro culturing of cells and tissues requires the supply of medium and nutrients. The culture environment should be stable in terms of pH, oxygen supply, and temperature. Cell culture media often comprise balanced salt solutions, amino acids, vitamins, fatty acids, and lipids to support the growth of the cells and/or tissues. The precise media formulations are often derived by optimizing the concentrations of every constituent. Different cell types are in need of different media compositions and/or cell culture conditions.

The three-dimensional cell culture template, as described herein, can be used to culture various cell types, alone or in co-culture and can be used with various types of cell culture media. In some embodiments, the cultured cells are eukaryotic cells, preferably mammalian cells. In preferred embodiments, the cultured cells are human primary or immortalized cells. Cells can be grown in adherent cultures or in suspension. In some embodiments, the cells are attached to the three-dimensional structure of the cell culture template.

Some cell types require surface modifications in order to attach properly to the material of the cell culture template. Surfaces may be coated prior to seeding the cells. Commonly used coating are collagen, fibronectin, and laminin In some embodiments, the cell culture template of the present invention can be used for many cell types without prior treatment or coating of the surface. The three-dimensional structures allow proper cell attachment without coating. However, if the coating is desired, the cell culture template with three-dimensional structures may be coated.

In some embodiments of the method for producing a cell culture template as described herein, the initial etched cavity in the monocrystalline substrate has access to the outside of the substrate defined by a pre-etching directional step. In preferred embodiments, the octahedral cavity in silicon has broad access to the outside of the substrate defined by a pre-etching directional step. In more preferred embodiments, the octahedral cavity in silicon has the widest point of the octahedral shape as opening and access to the outside of the substrate defined by a pre-etching directional step. When the etched cavity has broad access to the outside of the substrate, the production of multilevel three-dimensional structures is more optimal. FIG. 1 schematically displays the side view of etching an octahedron in a monocrystalline substrate. The top figure displays how the etched octahedron can have access to the outside of the substrate.

In some embodiments, the at least one three-dimensional structure of the cell culture template as described herein is produced using silicon as monocrystalline substrate. Thermal oxidation of silicon results in a layer of silicon oxide. In step 3 of the described method a layer of silicon dioxide is then grown and/or deposited. In the last step the bulk-silicon around the formed three-dimensional structure is removed. If the protective layer is created by thermal oxidation of the silicon, this will result in silicon oxide. Alternatively, if the protective layer is created by thermal nitridation of the silicon, this results in silicon nitride.

Silicon is a chemical element. Monocrystalline silicon can be used for the production of the three-dimensional structures as described herein. Monocrystalline silicon, is also called single-crystal silicon, in short mono c-Si or mono-Si. It consists of silicon in which the crystal lattice of the entire solid is continuous, unbroken to its edges, and free of any grain boundaries.

In some embodiments, the method for producing a cell culture template as described herein is used to produce three dimensional structures with closed or open apices. The three-dimensional structures can be produced with open apices when the last round of preparation is finished with creating apertures at all apices. In some embodiments, the open apices can be used to supply solutions to the cell culture. The three-dimensional structures can be produced with closed apices when the last round of preparation is finished with forming a protective layer, which also covers the apex or apices.

In some embodiments, the method for producing a cell culture template, as described herein, produces three-dimensional structures with higher complexity. To produce a structure with higher complexity steps, 6 to 8 of the method are repeated 2-10 times or higher, preferably 2-5 times. Each repeat of these steps results in an extra layer of octahedral structures, as exemplified between sequence FIG. 2C-2G. Each following layer will comprise smaller geometrical cavities. Preferably, each following layer will comprise smaller octahedrons at each apex of the previous layer.

In some embodiments, a subset of steps of the production method is repeated to create three-dimensional structures with a higher level of complexity (e.g., FIG. 2C-2G). After deposition of a protective layer to the etched geometrical cavity, an aperture is made at each apex of the outer layer of the geometrical shapes. The aperture is used to apply another round of anisotropic etching of at least one, or part of one geometrical feature of geometrical shape in the monocrystalline substrate. In preferred embodiments, the geometrical shape is an octahedron. The anisotropic etching is performed through the one or more apertures formed at the one or more apices. The new layer of geometrical cavities is subsequently protected with a protection layer.

Exemplary structures on a geometrical shape of octahedrons are shown in FIG. 2. FIG. 2C shows a simple three-dimensional structure that can be created with 1 round of anisotropic etching. FIG. 2D shows a three-dimensional structure that can be created with 2 rounds of anisotropic etching. FIG. 2E shows a three-dimensional structure that can be created with 3 rounds of anisotropic etching. FIG. 2F shows a three-dimensional structure that can be created with 4 rounds of anisotropic etching. And FIG. 2G shows a three-dimensional structure that can be created with 5 rounds of anisotropic etching.

In some embodiments, the method for producing a cell culture template as described herein, produces three-dimensional structures comprise a surface with a regular pattern of protrusions. These protrusions are built up from octahedral structures, and the octahedral structures are becoming narrower to the outside of the three-dimensional structure. The outside narrowing between structures is defined as the pitch. Among other factors, the pitch is determined by the three-dimensional level of complexity gained by the fractal generation.

The distance between the fractals can vary. The distance between the centers of any of two adjacent three-dimensional structures can also be called a pitch. Preferably the pitch between the three-dimensional structures is 5-100 μm, preferably 10-50 μm, more preferably 10-25 μm, most preferably 12-20 μm. The pitch between the three-dimensional structures depends on the placing, the orientation, and the size of the three-dimensional structures. For example, in preferred embodiments, the pitch between the three-dimensional structures placed in a hexagonal orientation is 12 μm, and the pitch between three-dimensional structures placed in a square orientation is 20 μm.

In some embodiments, the method for producing a cell culture template as described herein, comprises at least one three-dimensional structure having any of the following topographies:
a pyramid (G0, FIG. 2C),
a pyramid with on the apex an octahedral (G1, FIG. 2D),
a pyramid with on the apex an octahedral and on each apex of the octahedral a second level of octahedral structures (G2, FIG. 2E),
a pyramid with on the apex an octahedral and on each apex of the octahedral a second level of octahedral structures and on each apex of the second level a third level of octahedral structures (G3, FIG. 2F), or
a pyramid with on the apex an octahedral and on each apex of the octahedral a second levels of octahedral structures and on each apex of the second level a third level of octahedral structures and on each apex of the third level a fourth level of octahedral structures (G4, FIG. 2G).

The different level of complexities influences the surface pattern on the cell culture template. These patterns are more detailed when the three-dimensional structures have a higher level of complexity. When the level of complexity increases, the space between the three-dimensional structures may decrease.

In some embodiments, the at least one three-dimensional structure or the entire cell culture template comprising the three-dimensional structures are sterilized before growing cells. For example, the structures can be sterilized by chemical means, high temperature treament, irradiation, such as autoclave and UV light. In preferred embodiments, the three-dimensional structures or the entire cell culture template are sterilized by using UV, chemical means and/or high temperature treament.

In some embodiments the method for producing a cell culture template as described herein the at least one three-dimensional structure comprises multiple three-dimensional structures and wherein the multiple three-dimensional structures are placed in a lattice configuration. In preferred embodiments the structures are placed in a square or hexagonal lattice configuration, more preferably is a hexagonal orientation.

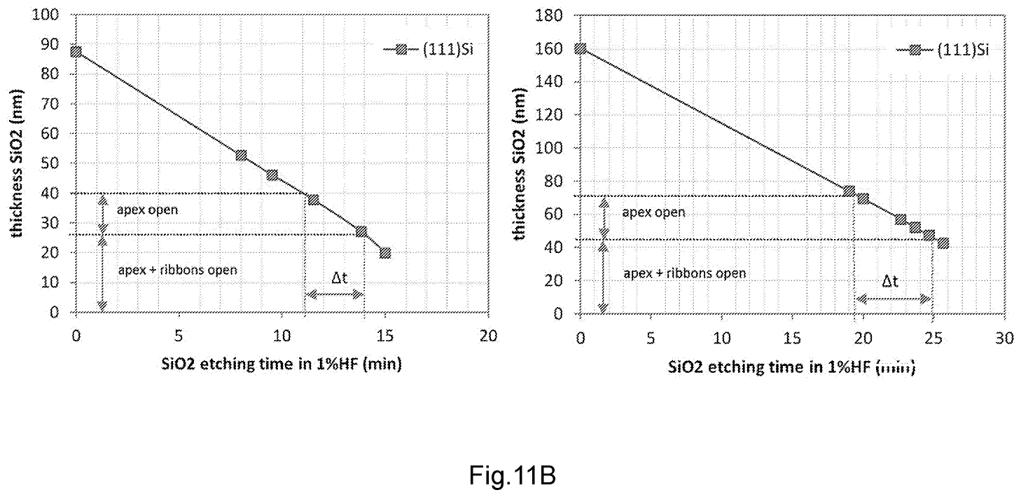

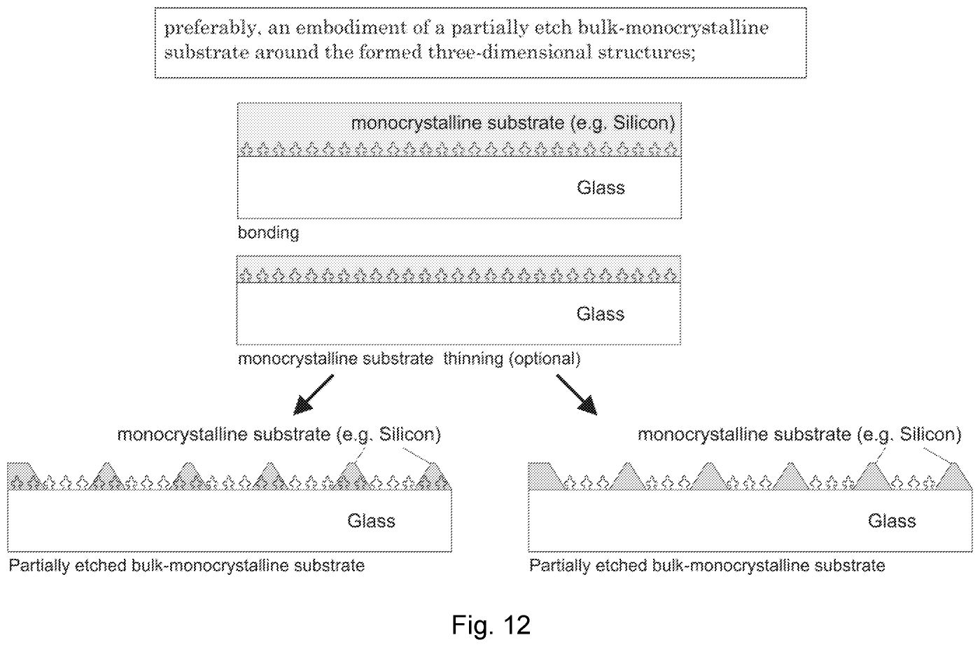

In some embodiments the method for producing a cell culture template as described herein comprises partial removal of the bulk-monocrystalline substrate. For this embodiment, the bulk-monocrystalline substrate is partially etched away around the multiple formed three-dimensional structures. In preferred embodiments, the bulk monocrystalline substrate is partially etched away in a manner to create multiple compartments, wherein the compartments comprise one or more three-dimensional structures. These compartments can be in the form of wells, by leaving rings of bulk-monocrystalline substrate unetched. The silicon rings will separate the wells and allow the wells to contain fluid. These wells are suitable to culture cells. Furthermore, structures of the left bulk-monocrystalline substrate can protect the fractal structures. The partial etching step is illustrated in FIG. 11.

The distance between the fractals can vary. The distance between the centers of any of two adjacent three-dimensional structures can also be called a pitch. Preferably the pitch between the three-dimensional structures is 5-100 μm, preferably 10-50 μm, more preferably 10-25 μm, most preferably 12-20 μm. The pitch between the three-dimensional structures depends on the placing, the orientation, and the size of the three-dimensional structures. In preferred embodiments, the pitch between the three-dimensional structures placed in a hexagonal orientation is 12 μm and the pitch between three-dimensional structures placed in a square orientation is 20 μm.

In some embodiments, the cell culture template, as described herein, further comprises at least one insulator. Insulators are made from material in which the electrons do not flow freely. As a result, very little electric current will flow through the insulator under the influence of an electric field. Amorphous silicon dioxide is a suitable material for an insulator. Therefore, the three-dimensional fractal structures, as described herein, can function as an insulator in the cell culture template. In preferred embodiments, the insulator is a three-dimensional structure of amorphous silicon dioxide.

In some embodiments, a method of the invention comprises a further comprise a step 9: providing the at least one three-dimensional structure with an inorganic layer, whereby the inorganic layer is in contact with the base three-dimensional material, I.e. the inorganic layer is provided to the surface of the at least one three-dimensional structure comprising the base three-dimensional material. Said step 9 is performed after step 5 and prior to providing the at least one three-dimensional structure with cells under growth permitting conditions to produce the cell culture template. Said inorganic layer are preferably provided by conformal deposition or by directional deposition. More preferably the inorganic layer is deposited on the base three-dimensional material using atomic layer deposition (ALD; for conformal deposition), physical vapour deposition (PVD) or sputtering (both for directional deposition). These techniques are well known in the art.

Said layer is provided to at least part of said three-dimensional structure, in particular a part of the structure that will be provided with the cells so that the cells will be cultured on the layer. Hence, in a preferred embodiment of a method of the invention, said method further comprises a a step 9: providing the at least one three-dimensional structure with an inorganic layer, whereby said step 9 is performed after step 5 and prior to providing the at least one three-dimensional structure with cells under growth permitting conditions to produce the cell culture template, and whereby said cells are provided to the at least part of the structure that is provided with said inorganic layer. I.e. said cells are provided to the surface of the at least on three-dimensional structure comprising the inorganic layer, in particular the cells are provided to the inorganic layer. Preferably, the cells are subsequently cultured on said layer.

Said part of the three-dimensional structure that is provided with the inorganic layer is preferably at least 25% of the surface area of the three-dimensional structure, and preferably the cells are subsequently provided to the at least part of the structure that is provided with said inorganic layer. More preferably at least 30%, more preferably at least 40%, more preferably at least 50%, more preferably at least 60%, more preferably at least 70%, more preferably at least 80%, more preferably at least 90% of the surface area of the of the three-dimensional structure.

In one embodiment essentially the entire surface of the three-dimensional structure is provided with the inorganic layer, and preferably the cells are subsequently provided to the at least part of the structure that is provided with said inorganic layer.

Said inorganic layer is compatible with cell culture.

In some embodiments, the inorganic layer preferably comprises platinum, gold, silver or a combination thereof.

In preferred embodiments, said inorganic layer allows for measurement by surface-enhanced Raman spectroscopy e.g. for high-resolutional molecule determination, electrical stimulation and recording e.g. of neuronal cells,

In some embodiments, the cell culture template, as described herein, further comprises at least one metal portion. Metal portions can provide other properties to the cell culture template, which can influence the cell culture.

In preferred embodiments, the metal portion is part of the three-dimensional structures of the cell culture template as described herein. The metal portion can be embedded and/or patterned on the three-dimensional structure. Metal portions can provide other properties to the three-dimensional structures, as described herein. Metal portions in the three-dimensional portions can facilitate an electric current. An electric current may influence the cells in culture. For example, an electric current may influence cell morphology and/or cell spreading a cell culture. Metal portions may also increase the flexibility of the three-dimensional structures.

In some embodiments, the metal portions in the cell culture template as described herein are used for external stimulation of the cells or tissues in culture. This external stimulation can be performed by means of three-dimensional structures. For example, external stimulation of cells and/or tissues in cell culture can be used to induce a synthesized rhythm in the waves.

A cell culture template comprising a three-dimensional structure and possibilities to perform external stimulation can be of great advantage for culturing muscle cells, especially cardiac muscle cells. Therefore, the cell culture template, as described herein, can improve muscle cell technologies and/or cardiac cell culture technologies. Furthermore, neurons and the synapses of neurons can be stimulated by an electric field or by a varying magnetic field. Therefore, the cell culture template, as described herein, can be used to culture neurons and/or neuronal tissues and simulate these cells during cell culture.

In some embodiments, the cell culture template, as described herein, uses electrodes for cell stimulation. In preferred embodiments, the three-dimensional structures can function as electrodes for cell stimulation. The cells in culture can be attached to the three-dimensional structures of the cell culture template. Therefore, stimulation via these structures will reach the cells directly. The direct contact contributes to a good transmission of the signals.

The disclosure further provides a cell culture template for growing and maintaining a cell culture, in particular a cell culture comprising primary cells. The cell culture template comprises cells seeded on a cell growth surface, for example a surface of an amorphous silicon dioxide. The surface is defined by at least one three-dimensional fractal structure carried on a support base, for example a layer of borosilicate glass.

The surface of the cell culture template may be defined by a multitude of, preferably at least almost identical, three-dimensional fractal structures evenly distributed on the support layer. In some embodiments, some of the three-dimensional fractal structures of the multitude of three-dimensional fractal structures on the support layer are covered by monocrystalline substrate with the other three-dimensional fractal structures of the multitude of three-dimensional fractal structures being exposed, i.e. free of monocrystalline, to form the cell growth surface. The monocrystalline substrate can be arranged to define one or more cell growth compartments having one or more exposed fractals. In some embodiments, the cell culture template has a lid is provided on a side of the cell layer opposite of the cell growth surface on top of and supported by the monocrystalline substrate.

The disclosure further provides a method for culturing cells or tissues comprising using the cell culture template produced by the method as disclosed herein and seeding cells, tissue and/or organoid structures, and culturing the seeded cell, tissue, or organoid.

Cells can be grown in adherent cultures or in suspension. In some embodiments, the cells are attached to the three-dimensional structures of the cell-culture template. The three-dimensional structures can increase the adhesion between the cell and the cell culture template. While not wishing to be bound by theory, adhesion of cells can provide signals which are needed for the growth and differentiation. Most primary cells require a surface to grow in vitro properly.

As demonstrated in the examples herein, the cell culture template produced by a method of the invention allows for the purification of primary fibroblasts or other motile cells in a single step. The purification takes place by a selective migration of motile cells, e.g. fibroblasts, into the free space of the template. This holds in particular for G1 and higher generations templates where motile tumor cells are excluded.

Different cell types require different cell culture conditions. Some cell types require surface modifications in order to attach to the material of the cell culture template properly. Surfaces may be coated prior to seeding the cells. Commonly used coating are collagen, fibronectin and laminin. The cell culture template of the present invention can be used for many cell types without prior treatment or coating of the surface. The three-dimensional structures allow proper cell attachment without coating. However, if the coating is desired, the cell culture template with three-dimensional structures may be coated.

The three-dimensional cell culture template, as described herein, can be used with various types of cell culture media.

In some embodiments, the cells are dissociated before seeding and culturing the cells in the cell culture template. Cells can be dissociated by known techniques, such as mechanical dissociation by pipetting or enzymatic dissociation by adding collagenase. Dissociated cells can be seeded as single cells in the cell culture template.

In some embodiments, the cells are seeded without further treatment as a multicellular tissue piece in the cell culture template.

In some embodiments, for the method of culturing cells as described herein, extra steps may be used to isolate specific cell types prior to seeding the cells in the cell culture template.

In some embodiments, the cells seeded in the cell culture template have also been cultured in another cell culture template prior to seeding in the cell culture template as described herein. For example, the cells may be cultured in suspension or a 2D cell culture template.

In preferred embodiments, the cultured cells or tissues are primary cells, preferably the cells are primary tissue cells. In some embodiments, the primary cells are primary tumor cells. In some embodiments, the cells are cancer-associated fibroblasts.

Primary cells are cells that are isolated directly from tissues. For example, these primary cells can be epithelial cells, fibroblasts, keratinocytes, melanocytes, endothelial cells, muscle cells, hematopoietic, and mesenchymal stem cells. The cultures can be heterogeneous. The cell culture can also be used to co-culture different cell types. In some embodiments, the primary cells cultured in the three-dimensional cell culture template are epithelial cells, fibroblasts, keratinocytes, melanocytes, endothelial cells, muscle cells, hematopoietic and/or mesenchymal stem cells. In some embodiments, the cultures are heterogeneous, comprising various cell types.

Furthermore, primary cells can be derived from healthy or diseased tissue, for example, tumors. Primary cells derived from tumors are called primary tumor cells. These cells can be tumor cells but also cells that are present in the microenvironment of the tumor and support the tumor cells. For example, cancer-associated fibroblasts. In some embodiments, the cultured cells are cancer-associated fibroblasts.

Primary cells are known to be very sensitive to their environment. In known culture templates, these cells need an additional supply of nutrients and/or other factors, for example, growth factors. These additional factors should be customized for each cell type. For example, endothelial cells have very different requirements than epithelial cells or neurons.

Although primary cells may be more difficult to work with, experiments using primary cells are thought to be more relevant and reflective to the in vivo environment. Primary cells retain the morphological and functional characteristics of their tissue of origin. Therefore, these cells can closely represent the human in vivo situation. For example, primary tumor preserves most tumor markers and known microRNAs.

The cell culture template comprising at least one three-dimensional structure as described herein can support the growth and survival of these primary cells. Although not wishing to be bound by theory, the material, shape and/or pattern of the three-dimensional culture template can support the primary tissue cells. The cell adapts its morphology to the spatial limitations of the three-dimensional structures. This can potentially activate the primary cells, for example, the cancer-associated fibroblasts, as shown in the experimental section.

Primary cells are known to have limited potential for self-renewal and differentiation. When these cells are cultured for a longer period, they show morphological and functional changes. The three-dimensional culture template, as described herein, can support the primary cells. Therefore, these cells will retain their tissue-specific characteristics for a longer period, which allows them to perform more extensive studies on these cells.

Cancer-associated fibroblasts are non-tumor cells that are present in the tumor microenvironment. The tumor-microenvironment is a multicellular tumor-supportive system and comprises cells from mesenchymal, endothelial and hematopoietic origin. The cells interact closely with the tumor cells and contribute to tumorigenesis. The tumor microenvironment is also a target for the development of anti-cancer drugs. Culturing cells from the tumor microenvironment, for example, tumor-associated fibroblasts is therefore of value for studies to tumor-targeting drugs.

In a preferred embodiment of the method for culturing cells or tissues as described herein, the cells are stem cells, preferably mesenchymal stem cells, adult stem cells, adipose adult stem cells and/or induced pluripotent stem cells. In some embodiments, the cells are progenitor cells. In preferred embodiments, the stem cells are not derived from embryones or embryonic tissue. Preferably, the stem cells are not embryonic stem cells.

Stem cells can self-renew and can differentiate into tissue-specific cells. Therefore, these cells have many applications, and there is a big interest in culturing stem cells and progenitor cells. The cell culture template comprising at least one three-dimensional structure, as described herein, can optimize the culture conditions for stem cells. Although not wishing to be bound by theory, the material, shape and/or pattern of the three-dimensional culture template can support the stem cells and allow them to differentiate specific cell types.

In some embodiments, the cell culture template, as described herein, can be used to grow or create functional 3D structures. In some embodiments, cells in the method for culturing as described herein form complex cellular assemblies, preferably a multicellular organoid.

An organoid is a miniaturized and simplified version of an organ produced in vitro in three dimensions. These organoids are multicellular and show realistic microanatomy. They are derived from one or a few cells from a tissue, stem cell, or introduced pluripotent stem cell. The cells in these organoids are organized and can be polarized, having an apical and a basal side. The three-dimensional structures of the described cell culture template can attribute to the formation of organoid structures and support these structures to grow.

In preferred embodiments, the shape, material and/or pattern of the three-dimensional structures of the culture template support the differentiation of the cells into tissue-specific cells and therefore stimulate the formation of the organoids. For example, patient-derived microtumors with bystander cells as an in vitro test for personalized chemotherapy. Neurospheres, the precursor of neurons to create transplants for spinal cord injuries and other neuronal damages, or neurological disorders.

In some embodiments, the cultured stem cells undergo differentiation when cultured in the tissue culture template comprising three-dimensional structures. In preferred embodiments, the cells undergo stem cell differentiation. The differentiation may be initiated by the shape, material and/or pattern of the three-dimensional structures. In preferred embodiments, the differentiation is initiated by the pyramidal shape and the pattern of the structures. For the pattern, the distance of the three dimensional structures is important.

In vitro culturing of cells and tissues requires the supply of medium and nutrients. The culture environment should be stable in terms of pH, oxygen supply, and temperature. Cell culture media often comprise balanced salt solutions, amino acids, vitamins, fatty acids and lipids to support the growth of the cells and/or tissues. The precise media formulations have often been derived by optimizing the concentrations of every constituent. Different cell types are in need of different media compositions. Furthermore, culturing of cells often requires the addition of serum. The serum is a complex mix of proteins, peptides, growth factors, and growth inhibitors. The most commonly used serum is fetal calf serum, which is used for a wide range of cell types. In addition, the medium may be supplemented with growth factors and cytokines.

During culturing, the cells use the nutrients supplied by the media and excrete their waste products into the media. Therefore, it is important to supply the cultured cells or tissues with fresh media regularly. The frequency of refreshing the media depends on the cell type and growth rate of the cells.

During the establishment of primary cultures, it is often necessary to include an antibiotic in the growth medium to inhibit contamination introduced from the host tissue.

After isolation, primary cells often undergo the process of senescence and stop dividing after a certain number of cell divisions or sense cell-cell contacts. It is challenging to retain the viability of primary cells. For the long-term viability of the cells, appropriate culture conditions are essential. Growth factors are often supplied by adding a serum to the culture medium.

In some embodiments of the method for culturing cells or tissues as described herein, the cultured cells are grown and/or be preserved in non-optimal growth conditions. At least one three-dimensional structure in the cell culture template supports the cultured cells. The three-dimensional structures provide a proper place to attach to. These circumstances allow to adapt to other culture conditions and still maintain the cell culture. Non-optimal growth conditions may comprise removal of certain factors from the culture medium, for example, growth factors. Non-optimal growth conditions may also comprise, maintaining the cell culture at room temperature instead of 37° C., low CO₂ (air) percentages instead of 5%, long-term growth of the cells, and/or less frequent medium change. As the cells also survive in non-optimal growth conditions, a cell culture platform as described herein is suitable for transport of living cells and cell cultures. During transport the cells remain healthy when transported outside an incubator

In some embodiments, the cell culture template comprising three-dimensional structures produced as described herein is composed of amorphous silicon dioxide and cells attached to the structure. Amorphous silicon dioxide is the non-crystalline form of silicon dioxide. It can be deposited in a thin film, but it can also provide a structure by itself. Amorphous silicon does not consist of small grains, also known as crystallites. In an amorphous structure, the atomic position is limited to short-range order only. In preferred embodiments, the three-dimensional structure of amorphous silica consists of SiO₂.

At least one three-dimensional structure of the cell culture template, as described herein, is suitable for microscopy purposes. Therefore, the cells can be analyzed while being attached to the three-dimensional structure.

### Definitions

As used herein, “to comprise” and its conjugations are used in its non-limiting sense to mean that items following the word are included, but items not specifically mentioned are not excluded. In addition, the verb “to consist” may be replaced by “to consist essentially of” meaning that a compound or adjunct compound as defined herein may comprise additional component(s) than the ones specifically identified, said additional component(s) not altering the unique characteristic of the invention.

The articles “a” and “an” are used herein to refer to one or to more than one (i.e., to at least one) of the grammatical object of the article. By way of example, “an element” means one element or more than one element.

The word “approximately” or “about” when used in association with a numerical value (approximately 10, about 10) preferably means that the value may be the given value of 10 more or less 1% of the value.

Features may be described herein as part of the same or separate aspects or embodiments of the present invention for the purpose of clarity and a concise description. It will be appreciated by the skilled person that the scope of the invention may include embodiments having combinations of all or some of the features described herein as part of the same or separate embodiments.

All patent and literature references cited in the present specification are hereby incorporated by reference in their entirety.

The invention is further explained in the following examples. These examples do not limit the scope of the invention, but merely serve to clarify the invention.

### EXAMPLES

### Example 1

Cell culture is the “working horse” toward a better understanding of biology in health and disease and as testing platform for toxicity and efficacy of new drugs. While the majority of results in biology and medicine is based on 2D cell culture, it is well known that 3D cell spheroids or multicellular organoid complexes are more realistic models. There are two major ways how to produce cell spheroids: i) floating cell spheroids in liquid or ii) cells embedded in hydrogels. To create floating spheroids, it is necessary to prevent the cell attachment to the culture dish surface. This can be achieved by increasing the surface hydrophobicity¹ or by polymer deposition²⁻⁴, prevention of attachment in general e.g. by hanging drop culture⁵ or by nano- or microstructuring of the surface⁶ (e.g. texturization of titan surfaces on implants⁷ or by deposition of polymeric nanomaterials^{3,8}). However, the structuring of the surfaces can also induce differentiation in stem cells⁶. Another form of inducing floating spheroids of stem cells by pelleting and hence clustering the cells in an Eppendorf cup was introduced by König and his group⁹. Attached or better embedded spheroids can be formed by seeding the cells in a hydrogel (e.g. Matrigel or other gels) or on a scaffold to form 3D spheroids.¹⁰ The main application in medicine of cell-repellent surfaces is to prevent bacterial attachment on implants or in odontology^{3,7,8} or the laboratory to study drug efficacy and toxicity in more realistic conditions. Both techniques have advantages and disadvantages. The floating spheroids are freely accessible for the exposure to drugs and released factors or extracellular vesicles can be easily collected. But the liquid cannot mimic the properties of surrounding tissue. The gel-embedded spheroids receive tissue-similar stimuli but collecting released factors as well as exposing them to a defined concentration of drug is difficult as also the surrounding gel interacts with the drug molecules and hence creating concentration gradients.

A new growth platform with periodically organized inorganic fractals of increasing complexity (G0-G4) is introduced. On this platform the cell growth of cancer-associated fibroblasts (CAF) isolated from patients with hepatocarcinoma and adipose stem cells on these fractal surfaces is studied. Our results indicate that some surface structures allow to grow cells in attached but free-standing 3D spheroids of CAFs and of stem cells. Other structures induce elongated cell growth in 2D with filopodia enwrapping the structures.

### Materials and Method

### Fractal Preparation

The fractal preparation follows the protocol described by Berenschot et al.⁸ The surfaces were structured in a hexagonal and a square orientation of the structures, which also varied in distance between fractals having a 12 and 20 μm pitch respectively. Scanning electron microscope (SEM) images of the fractals and the fractal-covered surfaces are shown in FIG. 2.

Because of the increasing size of the fractals, the free distances in the pitch decreases. In table 1 the size of the fractals and the free distances is shown.

TABLE 1
Fractal and surface features. Reported values in this table are in μm.

| | | | |
|---|---|---|---|
| | Length fractal | Free space | Free space |
| | (base/last | between | between |
| Generation | structure) | structures (calc.) | structures (meas.)* |

square

| | | | |
|---|---|---|---|
| G0 | 5.7 | 20 | 13.7 ± 0.05 |
| G1 | 5.8/5 | 19.9 | 13.5 ± 0.25 |
| G2 | 5.8/2.5 | 14.9 | 12 ± 0.18 |
| G3 | 6/1.2 | 12.5 | 10.4 ± 0.2 |
| G4 | 5.8/0.6 | 11.3 | 9.5 ± 0.15 |

hexagonal

| | | | |
|---|---|---|---|
| G0 | 6 | 12 | 6.2 ± 0.1 |
| G1 | 6.1/5.8 | 11.9 | 6.1 ± 0.15 |
| G2 | 6.2/2.5 | 6.9 | 4.3 ± 0.13 |
| G3 | 6.3/1.2 | 4.5 | 2.82 ± 0.24 |
| G4 | 6.2/0.6 | 3.3 | 2.46 ± 0.21 |

*Measured in SEM images by FIJI (ImageJ) analysis in 5 different positions.

### Cell Culture

All methods concerning the use of patient samples were performed in accordance with the relevant guidelines and regulations. The experiments were approved by a ethical committee. The patients signed an informed consent.

### Hepatocarcinoma Tissue and Cancer Associated Fibroblast (CAF) Isolation

Immediately after surgical resection, HCC tumor and peritumor specimens were cut into 0.5-1 cm pieces and left in MACS Tissue Storage Solution (130-100-008, Miltenyi). These tissue fragments were cut into smaller size pieces (1-2 mm), washed three times in Hanks balanced salt solution (HBSS), and then incubated in HBSS in the presence of collagenase Type IV (17104-019, Life Technologies) and 3 mM CaCl₂ at 37° C. under gentle rotation for 4 hours. At the end of this step the dissociation was mechanically facilitated by pipetting up-down the digested tissues with a large size orifice 50 ml pipet. The floating cells were collected and washed three times with HBSS and kept in this solution on ice (1^{st} digestion round). The decanted partially digested tissue specimens were subjected to a second round of digestion (as described above). The resulting dissociated cells (2^{nd} digestion round) were washed twice with HBSS, then combined with cells from 1^{st} digestion round, and centrifuged at 80 rcf for 5 minutes to separate epithelial and fibroblast cells. The fibroblasts contained in the supernatant were centrifuged at 100×g for 10 minutes, and the fibroblasts in the pellet were purified through positive selection using anti-fibroblasts MicroBeads and the MS Column (Miltenyi Biotech), according to the manufacturer's instructions. CAFs were then cultured in IMDM+20% FBS. To assess the purity of CAFs preparation, immunofluorescence or flow cytometry analyses were performed to evaluate the expression of mesenchymal markers, such as vimentin and smooth muscle actin alpha (αSMA). The presence of minimal contaminating non-fibroblastic cells (mostly cancerous hepatocytes, cholangiocytes and macrophages) was evaluated by using antibodies to EpCAM, CD45, and CD11b.

CAFs were trypsinized and resuspended in complete DMEM medium at the concentration of 4×10⁵ cells/ml. 50 μl of cell suspension (containing 2×10⁴ cells) were seeded in triplicate onto the fractal surface coated templates (1×1 cm; control (flat silicon, G0-4, square and ehexagonal orientation) placed in 6-well plates (3 in one well).

First the cells were incubated for 4 hours at 37° C. and 5% CO₂ without additional medium in order to allow them to attach exclusively onto the fractal coated surfaces to have a define number of cells. Then the templates were covered with 3 ml of complete medium and placed in the incubator, changing the medium every 3 days.

At day 8 and day 13 one template for each sample was fixed for 10 minutes with 4% paraformaldehyde in phosphate buffered saline (PBS) at pH=7.4. The fixed cells were stored at +4° C. for further use.

Human Adipose Stem Cells (hADSC)

The cell culture for the hADSC followed the protocol described by Legzdina et al.¹². In brief, cells were grown in DMEM/F12 medium (Euroclone, Italy) containing 10% fetal bovine serum (FBS) (Euroclone, Italy), 20 ng/ml basic fibroblast growth factor (bFGF) (Lonza Sales, Switzerland), 2 mM L-glutamine and 100 μ/ml:100 μg/m penicillin-streptomycin and cultured in a humidified atmosphere at 37° C., 5% CO₂. Medium was replaced every third day.

### COLO 205 Cells

The human colon adenocarcinoma derived from metastatic site: ascites, COLO 205 cell line (ATCC® CCL-222™, ™, LGC Standards S.r.l., Italy) was cultured in RPMI-1640 medium (Euroclone, Italy) with foetal bovine serum (FBS South America, Euroclone, Italy) to a final concentration of 10%, 2 mM glutamine (Euroclone, Italy), and 1% penicillin/streptomycin (Euroclone, Italy). Cells were cultured at 37° C. in humidified atmosphere containing 5% CO₂.

HLF cells

HLF (JCRB Cell Bank, JCRB0405, Osaka, Japan) is a non-differentiated hepatocarcinoma cell line. The cells were cultured in DMEM medium (Gibco), supplemented with 10% FBS, 1 mM pyruvate, 25 mM HEPES, 100 U/ml penicillin-streptomycin and maintained at 37° C. in atmosphere containing 5% CO₂.

### Culture on the Fractal Substrate

Three fractal coated templates (1 cm×1 cm) were placed in 6-well plates if the experiment was in triplicate or in a 24-well plate if only 1 template was used and sterilized by irradiation with UV-light in the laminar flow hood for 1 h. The 2D cultured cells were trypsinized and resuspended in complete DMEM medium at the concentration of 4×10⁵cells/mL. 50 μL of cell suspension (containing 2×10⁴ cells) were seeded on the sterile substrates. Each experiment was performed in triplicate. First, the single cells were incubated for 4 h at 37° C. and 5% CO₂ without additional medium in order to allow them to attach exclusively onto the fractal coated surfaces to have a defined number of cells. Then the substrates were covered with 3 mL of complete medium and placed in the incubator, changing the medium every 3 days. The isolate from primary CAF preparation was grown for 8 and 13 days, then fixed for 10 minutes with 4% paraformaldehyde in phosphate buffered saline (PBS) at pH=7.4 and then treated for immunohistochemistry. The HLF cells were fixed and stained after 4 days of culture on the fractal surfaces.

Respective CAF cells were grown as control on treated 24-well plate (Corning Cellbind Surface) except for the HLF where the cell growth was compared to cells grown in Matrigel.

### Culture in Matrigel

Thirty μl of Matrigel (Corning Inc., USA) were layered on the bottom of wells of a 96-well plate and jellified for 20 min in the cell culture incubator (37° C., 5% CO₂). One thousand hepatocellular carcinoma HLF cells were mixed with additional 30 μl of Matrigel, layered on the first Matrigel gel layer and left for additional 20 min in the incubator. Finally, 90 μl of complete DMEM medium were added to the Matrigel embedded cells and the cells were allowed to grow for 13 days to form spheroidal multicellular structures. The medium was replaced every 2 days.

### Proliferation and Adhesion to Fractal Surfaces

Proliferation was evaluated by cell counting in a Burker chamber, hADSC were seeded with a density of 1.4×10⁴ cells/well, COLO 205 cells 1×10⁴. Both cell lines were grown on six different fractal templates in 24-well plates in complete medium at 37° C. in 5% CO₂, the control condition was represented by cells seeded directly on a well of 24-well plates. After 24 h cells were then extensively washed in phosphate-buffered saline (PBS) detached with Trypsin/EDTA and counted. Values were expressed as the absolute number of cells or as percent variation with respect to basal number, ±s.d. After 2, 24, 48, and 96 hours, the cells were observed and photographed to document any differences in proliferation and adhesive capacity. Each experimental point was repeated 3 times.

### Flow Cytometry

Analysis of markers to detect HCC cancer cells and CAFs was performed using the following anti-human antibodies: Alexa Fluor 488-conjugated IgG2a to alpha-fetoprotein (AFP, BD Biosciences, USA); FITC-conjugated IgG1 to CD13 (Merck, Germany); FITC-conjugated IgG2b to CD44 (BD Biosciences, USA); FITC-conjugated IgG1 to CD90 (BD Biosciences, USA); FITC-conjugated IgG1 IgG1 to CD133 (Miltenyi Biotec, Germany); Unconjugated IgG1 to CD151 (abeam, UK); FITC-conjugated IgG2b to EpCAM (BioLegend, USA); Unconjugated IgG1 to OV-6 (R&D Systems, USA); FITC-conjugated IgG1, IgG2a and IgG2b isotype control antibodies (Miltenyi Biotec, Germany); Alexa Fluor 488-conjugated IgG isotype control antibody (abeam, UK); Alexa Fluor 488-conjugated anti-mouse antibody.

Briefly, the cells were detached using StemPro Accutase Cell Dissociation Reagent (Thermo Fisher Scientific, USA) and incubated with fluorophore-conjugated antibodies for surface staining of CD13, CD44, CD90, CD133, CD151, EpCAM and OV-6 for 1 hour at 4° C. in the dark. For AFP staining, cells were fixed and permeabilized using Foxp3/Transcription Factor Fixation/Permeabilization Concentrate and Diluent (eBioscience, Thermo Fisher Scientific, USA), prior to antibodies incubation. A second incubation step with secondary Alexa Fluor 488-conjugated antibody (for 1 hour at 4° C. in the dark) was performed to detect CD151 and OV-6. Fluorophore-conjugated isotype antibodies were used as controls related to detection of AFP, CD13, CD44, CD90, CD133, EpCAM. Alexa Fluor 488-conjugated anti-mouse antibody was used as control related to detection of CD151 and OV-6. Cells were analyzed using the Navios flow cytometer and the data were processed using the software Kaluza (Beckman Coulter).

### Fluorescence Microscopy

For the fluorescence imaging, the fixed cells were permeabilized with 0.1% Triton X-100 in PBS (2% bovine serum albumin added) for 15 minutes, and then incubated for 1-2 hours in the presence of Phalloidin-Tetramethylrhodamine B isothiocyanate (TRITC; Sigma-Aldrich) to visualize the actin cytoskeleton.

To distinguish CAFs from tumor cells, the cells were stained with AFP antibodies covalently bound to Alexa Fluor488 (tumor) and for α-smooth muscle actin (α-SMA; CAF). Detection of α-SMA and α-fetoprotein expression by immunofluorescence imaging was performed on 4% Paraformaldehyde-fixed cells. Fixed cells were permeabilized with 0.1% Triton X-100 in PBS for 10 minutes. Cells were washed three times with PBS and then incubated with 1% BSA in PBS (PBS+0.1% Tween 20) for 30 min to block unspecific binding of the antibodies and thereafter incubated with the diluted antibodies in 1% BSA in PBS overnight at 4° C. (α-SMA: Cell Signalling Technology, 1:100; AFP: BD Pharmingen, 1:100). The cells were washed three times in PBS, and for α-SMA, they were incubated with a secondary Antibody Alexa Fluor® 488 conjugate (Invitrogen) diluted in 1% BSA in PBS (1:50) for 1 h at room temperature in the dark.

After three washes with PBS the surfaces of templates with adhered cells were covered with 4′,6-diamidino-2-phenylindole (DAPI)-supplemented antifade mounting medium (VECTASHIELD, Vectorlabs). Additionally, the cells were stained with an anti-Focal adhesion kinase 1 (FAK) antibody which was covalently bound to a quantum dot emitting at 585 nm (SiteClick™ Qdot™ 585 Antibody Labeling Kit; ThermoFisher; ordering no. S10451). The FAK-antibodies were labeled following the modified protocol of the distributor.

### Light Microscopy

COLO 205 and hADSC cells were visualized by means of an OLYMPUS CKX41 microscope with a 4×/0.25 PHP objective.

### Results and Discussion

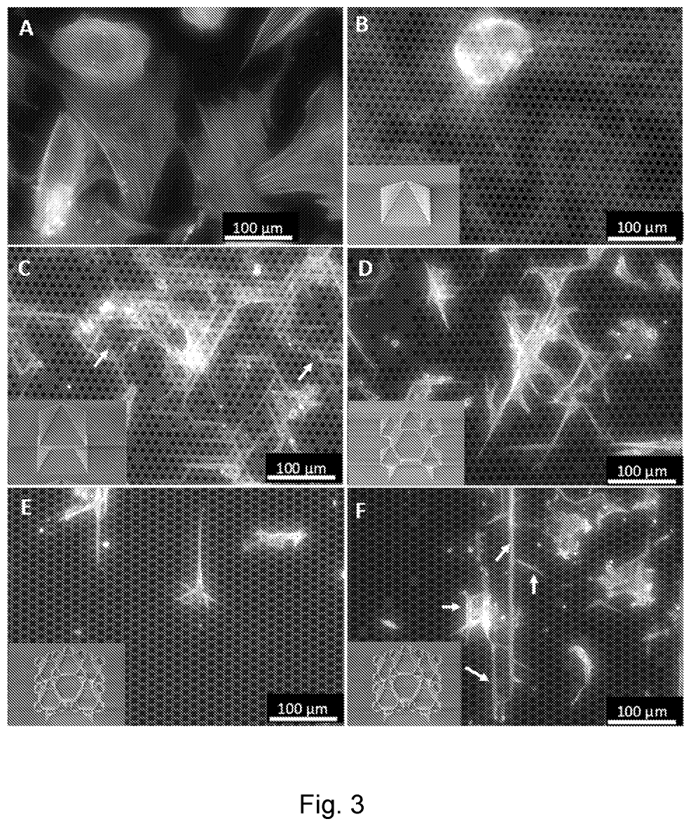

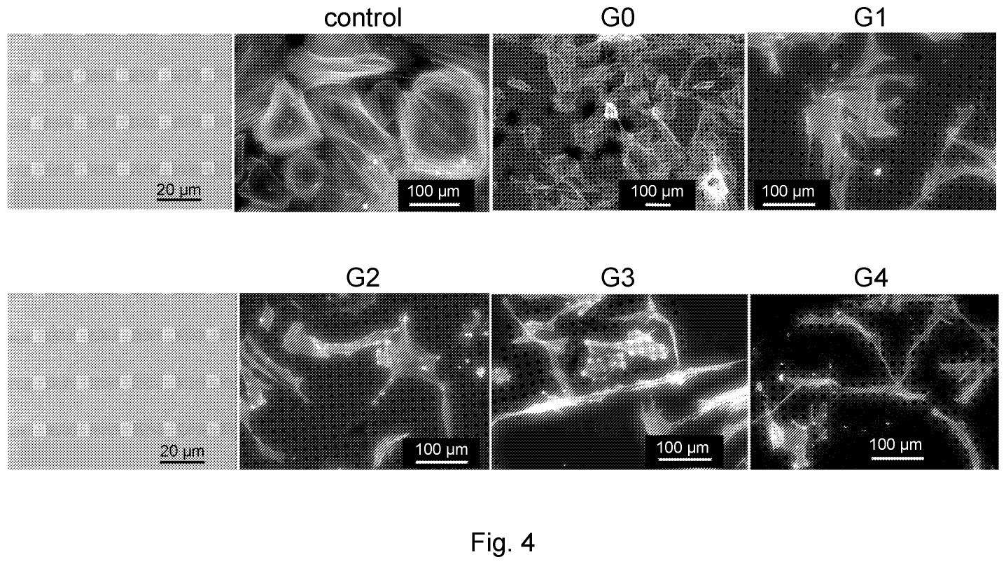

The fractal preparation follows the protocol described by Berenschot et al.¹¹ Inorganic fractal structures were periodically deposited on a glass surface, sterilized by a simple exposure for 1 h under the UV light in the laminar flow cabinet, and without any further treatment the primary CAF cells were seeded on the different templates. The isolated primary cancer-associated fibroblasts (CAFs) from hepatocarcinoma patient were seeded on fractal substrates of different generations and lattice configurations with a cell density of 2×10⁴ cells. The template size was 1 cm×1 cm for all generation (G0-G4) and flat etched SiO₂ grown on silicon and bonded/back etched (flat SiO₂). The templates were placed in a 24-well plate without additionally functionalization (e.g. extracellular matrix molecule addition). They were sterilized by UV exposure for 1 h immediately prior use. Plastic and flat SiO₂ were used as controls. In order to have a defined number of cells on the template, the cells were left to attach for 4 h before the wells were filled with medium. Their growth and morphology were monitored daily by microscopic inspection. On day 8 and day 13, the cells were fixed and fluorescently stained by DAPI to visualize the nucleus and by TRITC-phalloidin for the actin filaments of the cytoskeleton. Representative images for the CAFs on the hexagonal oriented templates on day 13 are shown in FIG. 3. The CAFs on the square configuration 8 days after seeding can be found in FIG. 4.

In the following we will describe some interesting features observed for the different cells grown on the surfaces covered by periodically repeating fractals (FIGS. 3 and 4).

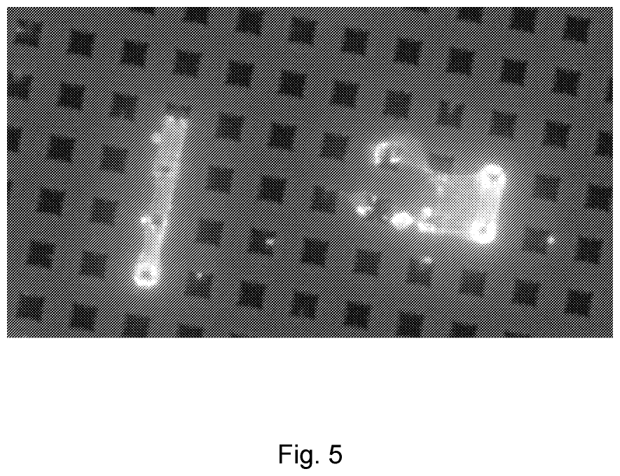

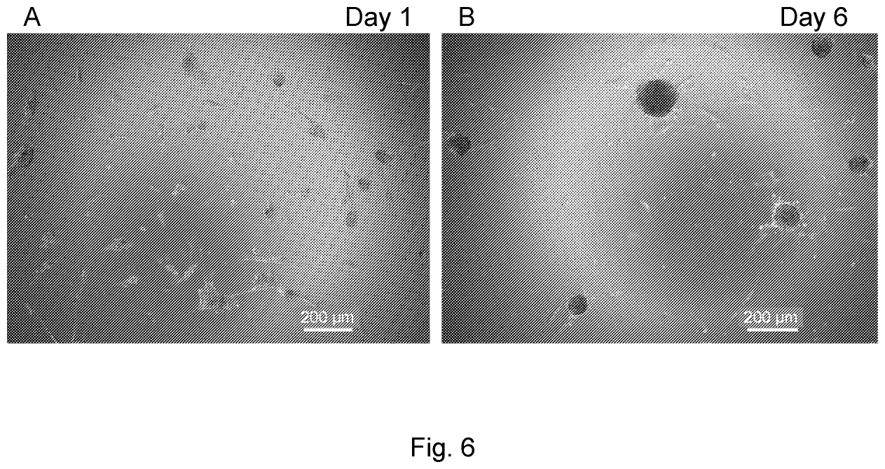

In general, it can be observed that the surface area covered by single cells is higher for the square configuration than for the hexagonal one. Little difference can be seen between the morphology of the cells on day 8 and day 15. The CAFs on the square configuration appear round while the cells on the hexagonal configuration are elongated with even elongated nuclei (arrows in FIG. 3C and F) and develop well-connected lamellipodia. While the nuclei are usually located between the fractals it is obvious that the lamellipodia are actively interacting with the fractals indicated by the high concentration in actin (red signal in FIG. 5)

Detailed cellular studies about the influence of the fractal microstructures on cell morphology, proliferation, viability, differentiation, and activation for each cell type (CAF, stem cells, COLO205) are ongoing and are scope of future publication.

### Spheroidal Cell Growth

The most interesting result of the Fractals coated surfaces as cell growth platform was the presence of spheroidal cell clusters by the CAFs isolated from hepatocarcinoma tissue of patients (FIGS. 3 and 4). CAFs grown on flat silicon surfaces sometimes and on G0 of both configuration always show a 2D layer of fibroblast-like cells directly attached to the fractals and in some regions 3D spheroidal cell clusters of a diameter of >100 μm attached to this 2D cell layer (FIGS. 3B, 7 and 5). Usually 16-20 spheroids were observed per 1×1 cm template for both configurations of G0 and on the control consisting of a flat amorphous SiO2 surface. We observed that the precursors of the spheroids already form on day 1 after seeding the single cells (FIG. 6A) which then grow into dense large spheroids within 8 days (FIG. 6B). Larger spheroidal cell clusters show only a diffuse blue fluorescence signal in the interior indicative of the absence of defined nuclei. We assume that it is a necrotic core surrounded by layer of intact cells.

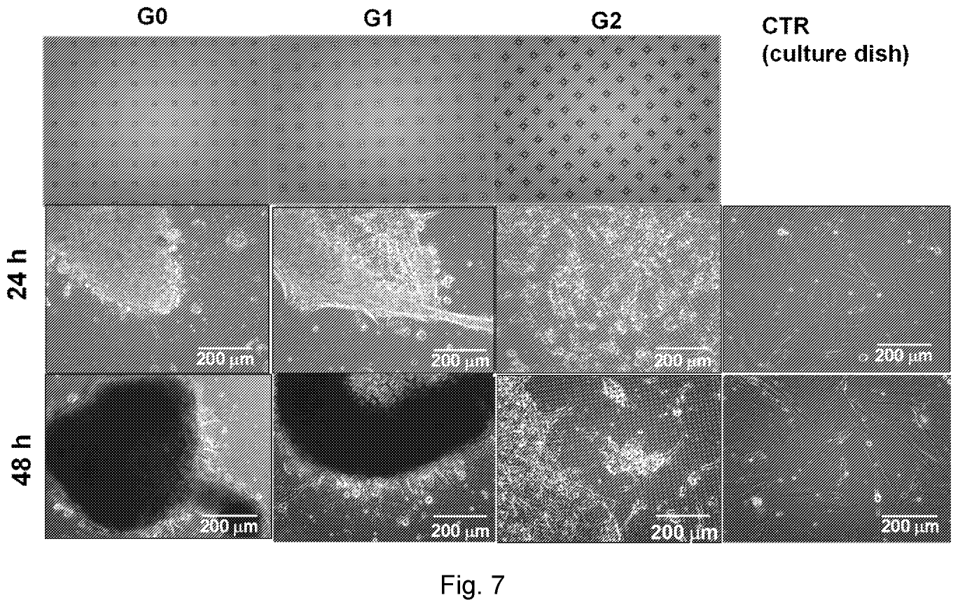

Interestingly, the same result was observed for hADSC as it can be seen in FIG. 7 for the square configuration.

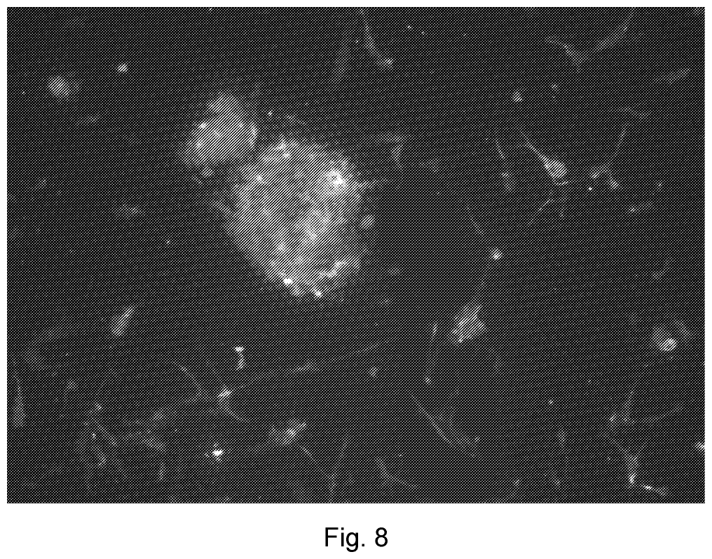

Firstly, a higher number of cells were detected on the fractal coated template as compared to the plastic surface of a cell culture dish. However, counting the cells was not straightforward as on the higher generations (G3,4) the cells were more difficult to detach by trypsinization. After 24 h clusters of cells are forming on G0 and G1 square configuration while on G2 a cell layer can be observed. The cluster form dense spheroids after 48 h as it can be seen in the lower panel in FIG. 7 (lower panel). On the hexagonal configuration we observed even on the G2 templates hADSC spheroids and differently to the CAF spheroids intact cells (fluorescence image: nuclei stained with DAPI (blue); CD90, a biomarker for stem cells as well as neurons stained with FITC-labelled anti-CD90 antibody (green)) can be found in the interior of the spheroids. A detailed investigation confirmed that the fractal surfaces induce a differentiation into nestin-positive neurospheres (FIG. 8).

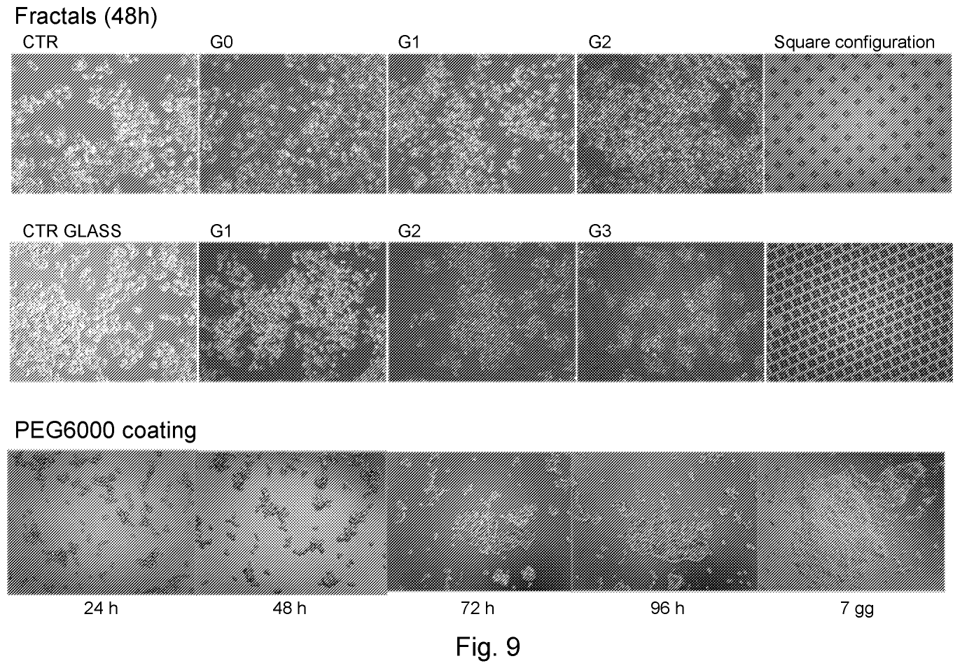

In contrast, no spheroidal growth was seen for the colon adenocarcinoma cell line, COLO205. The COLO205 was growing in 2D on all tested surfaces (FIG. 9 upper panels; G0-G3, both configurations) for up to 96 h. This is in good agreement with our finding that COLO205 in general do not form spheroids even in other spheroid producing system (FIG. 9 lower panel) following a cell repellent PEG6000 coating⁴. CAFs on the Hex lattice configuration appear as stellate-like cells with even elongated nuclei and with well-developed lamellipodia connected to the fractal structures. The cell nuclei are mainly located between the fractals while lamellipodia interact with the fractals as indicated by the high concentration in actin (red signal in FIG. 5). A detailed study about the trigger induced by the fractals on cell morphology, proliferation, viability, proteomics and genomics of primary cells is on-going and are the scope of future publications.

On different fractal surfaces we observed different responses as it is summarized in the table in table 2.

TABLE 2
Summary of all tested cells, tissues and cell lines and there results
on the different fractal surfaces

| | | | | | |
|---|---|---|---|---|---|
| Cell type/ | | | | | |
| Generation | G0 | G1 | G2 | G3 | G4 |

Differentiation

| | | | | | |
|---|---|---|---|---|---|
| Adipose- | 3D | 3D | 3D | 2D | 2D |
| derived stem | | | | | |
| cells | | | | | |
| HT29 | 2D | 2D | | | |

2D/3D cell culture

| | | | | | |
|---|---|---|---|---|---|
| Primary | | | | | |
| tumor + | | | | | |
| Cancer- | | | | | |
| associated | | | | | |
| fibroblast | | | | | |
| pancreas | 2D/3D | | | | |
| hepatocarcinoma | 2D/3D | 2D CAF | 2D CAF | 2D CAF | 2D CAF |
| Cell lines | | | | | |
| Caco2 | 3D | 3D | 3D | 3D | 3D |
| COLO 205 | 2D | 2D | 2D | | |
| HLF | 3D | 3D | 3D | 3D | 3D |

1-step purification

| | | | | |
|---|---|---|---|---|
| Cancer- | 2D | 2D | 2D | 2D |
| associated | | | | |
| fibroblasts | | | | |

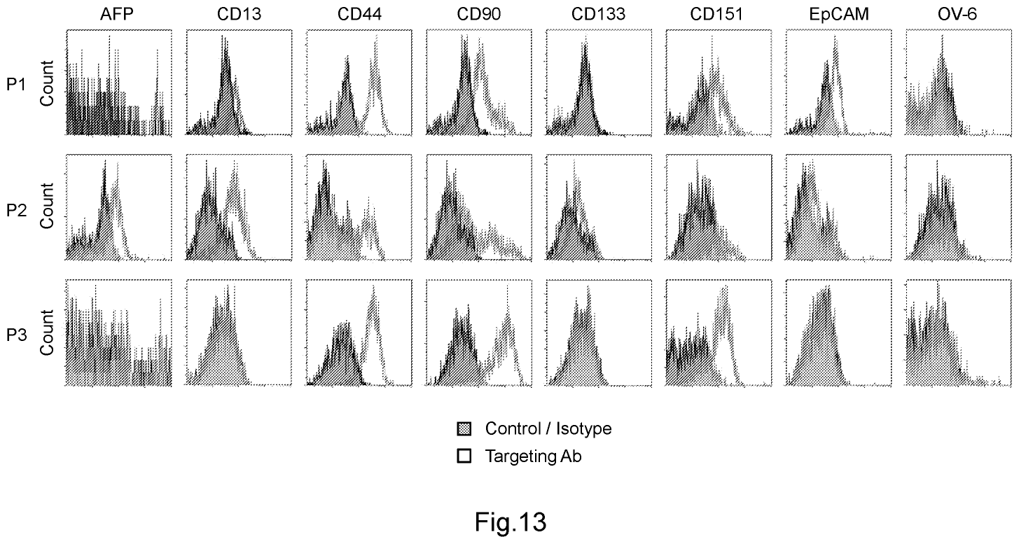

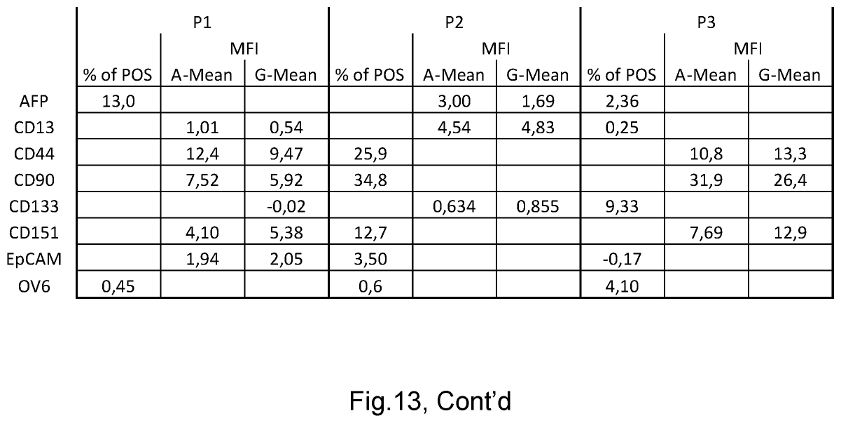

To understand the origin of the spheroid forming cell in case of the CAF isolate, we analyzed CAF isolates from 2D cell culture by FACS for biomarkers of different cell types (FIG. 13).

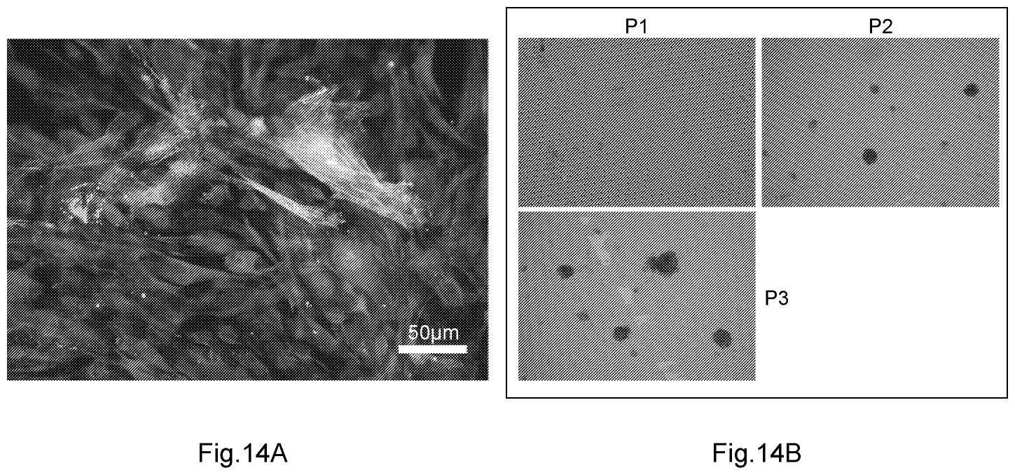

The isolation of CAF cells contains different amounts of cells positive for biomarkers for cancer stem cells (tumor stem cells; CD13[14,15], 44, 90[15], 133[16], OV6[15]), epithelial cells (EpCAM[15]), or general tumor cells (AFP[13]). This can be seen also in FIG. 14A where only approx. 20% of the cells are positive for α-SMA (CAF) when cultured in 2D. If the cells isolated from 3 patients and characterized by FACS (FIG. 13) are cultured for 6 days on G0Hex interestingly those of patient 2 and 3 are forming spheroids (FIG. 14B)

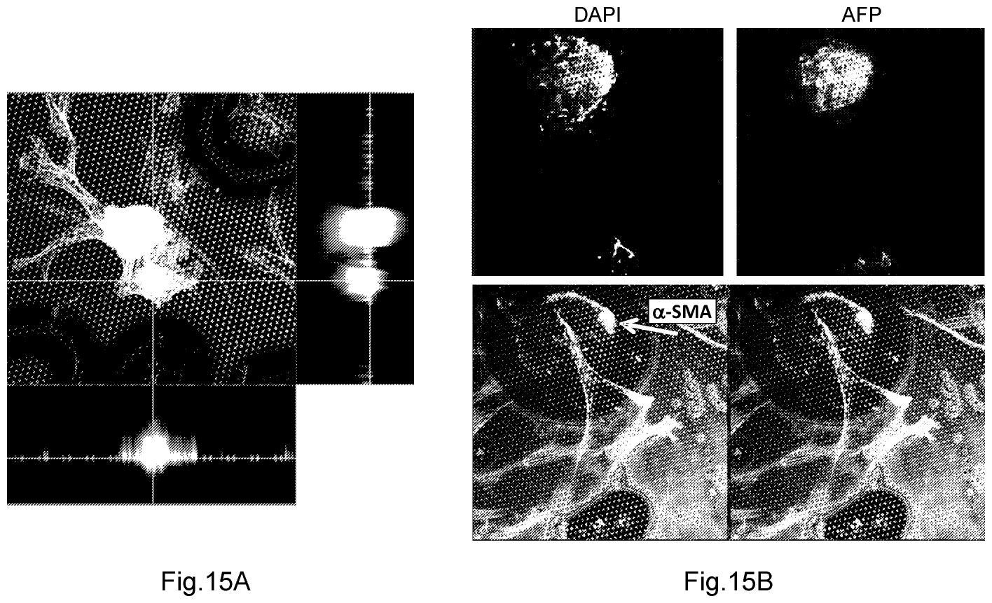

Z-stacks of spheroids by confocal microscopy confirmed the 2D layer of α-SMA positive CAFs cells and the spheroidal form of the microtumors (FIG. 15A). Interestingly, it seems that the 2D layer is situated on the level of the center of the spheroid (50 μm from the top and the bottom). This is surprising as the height of the fractals is only 15 μm. To understand if the spheroids digest the amorphous silica layer the organic material (cells) were etched by piranha solution and the underlying surface was visualized by optical microscopy and electronic microscopy. No changes of the inorganic surface can be observed (data not shown). Fractals interact with the light and induce a change of refractive index therefore the fraction of the microtumor embedded within the fractals seems distorted and enlarged.

The microtumors were then co-stained with AFP (green) and α-SMA (red) antibodies. The images in FIG. 15B showed that a capsule of fibroblasts encloses in a microtumor positive for AFP. No AFP signal can be seen in the 2D layer confirming that this layer consist exclusively of CAFs.

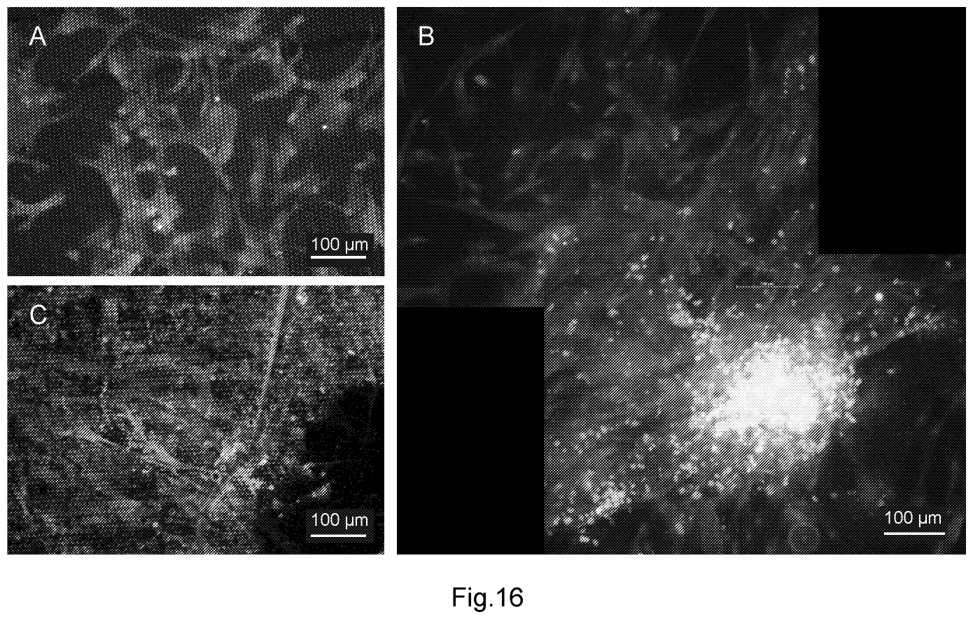

While the G0 templates foster the growth of complex 2D-tumor spheroid cluster, the tumor cells seem to be excluded from the templates for G1 and higher generations (FIG. 3C-F). In order to explore if these templates can be used to isolate CAFs in a one step process, pieces of peritumoral and tumoral tissue were placed on the fractal templates. After 2-3 days, first stellate CAF-like cells start to migrate into the fractal template. After 20 days, the tissue was removed and a layer of cells which invaded the fractal surface was stained for AFP and α-SMA (FIG. 16).

Neither the peritumoral tissue (no tumor cells) on G0 (FIG. 16A) nor the tumoral tissue on G1 (FIG. 16C) show any green fluorescence for AFP. In both cases only a 2D cell layer positive for α-SMA (red) can be seen. In contrast, if the tumor piece is in contact with a G0, a strong yellow signal for green AFP co-localized with red α-SMA antibodies can be detected where the tumor was in contact with the G0Sqr fractal template and later mechanically removed. Moreover, a gradual decline in AFP signal was observed in more distant cells. The spot-like appearance of the AFP signal can indicate that it stems from invadosomes which are known to be enriched in actin and penetrating the microenvironment.

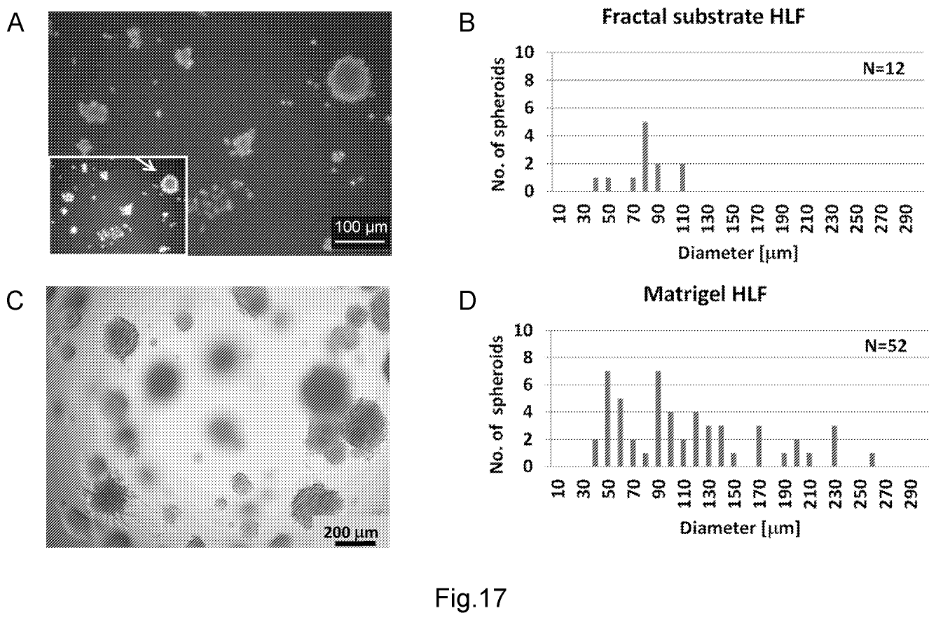

Finally, in absence of fibroblast as e.g. for cancer cell lines, G0 fractal templates induce a fast formation of spheroids as it can be seen exemplarily in FIG. 17A for HLF, a hepatocarcinoma cell line. The spheroid formation was compared to the growth of HLF cells in Matrigel (FIG. 17B).

It is noteworthy that spheroids of comparable size grow on the templates in 4 days while they needs 13 days in Matrigel. The average size of spheroids on the fractal substrate after day 4 was 74±20 μm (N=12), while the size of spheroids grown in Matrigel was 108±57 μm (N=52) at day 13. A direct comparison on day 4 is not possible because the spheroid growth in Matrigel starts with embedded single cells and the growth is exponential (12). Therefore, it is expected that only small clusters of few cells have formed on day 4. The HLF cells on the fractals were also seeded as single cells but already in the process of attachment they start clustering as it can be seen for CAFs in FIG. 6.

### Conclusion

A novel cell growth platform is introduced. This platform is especially suitable for difficult to grow cells such as stem cells and primary cells (CAFs and tumor cells). These templates coated with periodical fractal structures are easy to sterilize as it consists of inorganic material. Without any further treatment or functionalization, such as deposition of extracellular matrix molecules, it enhanced the complex 2D spheroidal growth of cancer-associated fibroblasts from patient samples. For some structures, a selective growth of isolated CAFs and suppression of the growth of the contaminating tumor cells was observed, which cannot be avoided in CAF isolation. However, if the co-culture of tumor cells in association with the CAFs is necessary, e.g., for testing different therapeutic options on microtumors to optimize the treatment for the patient, other fractal structures were found to support the growth or survival of 3D microtumors. These microtumors were found after 8 days of culture and provide a more realistic model of the patient's tumor than 2D isolated tumor cells. For G1-4 fractal surfaces, we observed a selective growth of CAFs which allows a one-step CAF isolation directly from the tumor. In tumor cell lines we observed an enhanced spheroidal cell growth as compared to the standard 3D matrix growth system.

### REFERENCES

1. Neto, A. I., Levkin, P. A. & Mano, J. F. Patterned superhydrophobic surfaces to process and characterize biomaterials and 3D cell culture. Mater. Horizons 5, 379-393 (2018).
2. Uhlig, K. et al. On the influence of the architecture of poly(ethylene glycol)-based thermoresponsive polymers on cell adhesion. Biomicrofluidics 6, 24129 (2012).
3. Hoffmann, J. et al. Blood cell and plasma protein repellent properties of Star-PEG-modified surfaces. J. Biomater. Sci. Polym. Ed. 17, 985-996 (2006).
4. Serrati, S. et al. Reproducibility warning: The curious case of polyethylene glycol 6000 and spheroid cell culture. PLoS One 15, 1-13 (2020).
5. Timmins, N. E. & Nielsen, L. K. Generation of multicellular tumor spheroids by the hanging-drop method. Methods Mol. Med. 140, 141-51 (2007).
6. Engel, E. et al. Mesenchymal stem cell differentiation on microstructured poly (methyl methacrylate) substrates. Ann. Anat. 191, 136-144 (2009).
7. Rosales-Leal, J. I. et al. Effect of roughness, wettability and morphology of engineered titanium surfaces on osteoblast-like cell adhesion. Colloids Surfaces A Physicochem. Eng. Asp. 365, 222-229 (2010).
8. Zhang, N. et al. Nanostructured Polymeric Materials with Protein-Repellent and Anti-Caries Properties for Dental Applications. Nanomaterials 8, 393 (2018).
9. König, K., Uchugonova, A. & Gorjup, E. Multiphoton fluorescence lifetime imaging of 3D-stem cell spheroids during differentiation. Microsc. Res. Tech. 74, 9-17 (2011).
10. Griffith, L. G. & Swartz, M. A. Capturing complex 3D tissue physiology in vitro. Nat. Rev. Mol. Cell Biol. 7, 211-24 (2006).
11. Berenschot, J. W. et al. 3D-fractal engineering based on oxide-only corner lithography. Symp. Des. Test, Integr. Packag. MEMS/MOEMS, DTIP 2016 3-6 (2016). doi:10.1109/DTIP.2016.7514895
12. de Hoogt, R. et al. Protocols and characterization data for 2D, 3D, and slice-based tumor models from the PREDECT project. Sci. Data 4, 170170 (2017)

### Example 2 (Berenschot et al. 2016)

An exemplary preparation of three-dimensional fractal structures

In order to be able to fabricate 3D fractals with oxide-only corner lithography, the grown amorphous silicon dioxide layer should be conformal on convex corners as well as equally thick on the silicon (100) and (111) crystal planes. If these requirements are not fulfilled, the layer of SiO₂ cannot be properly patterned by means of time-stopped isotropic etching (i.e., due to thickness variations, the SiO2 is removed from locations where it should remain), or will not function as a proper mask during selective anisotropic etching of silicon. Therefore, this simplified process uses (dry) thermal oxidation at 1100° C. Oxidation of silicon at this temperature leads to fundamental differences in the grown oxide compared to thermal oxidation at relatively low temperatures (≤950° C.), in terms of layer thickness on (100) and (111)-silicon crystal planes as well as layer conformality around convex corners.

At low thermal oxidation temperatures (≤950° C.) the oxide thickness at convex and concave corners is thinner than a flat (100)-Si planes due to compressive stress at the corner structures [5], [6]. At temperatures of 1000° C. the formed oxide layer on convex corners is not thinned with respect to the layer thickness on planar (100)-Si, but at this temperature, there is a difference in oxide growth rate on the main crystal directions of silicon [7]. Upon dry thermal oxidation of silicon at 1100° C. the mentioned aspects regarding non-conformality on convex corners and differences in oxide layer thickness on (100) and (111) Si-planes are avoided [7]. In concave corners, the severe compressive stress that develops [8] does not relief, and the connected reduction in the oxidation rate leads to a locally thinner layer.

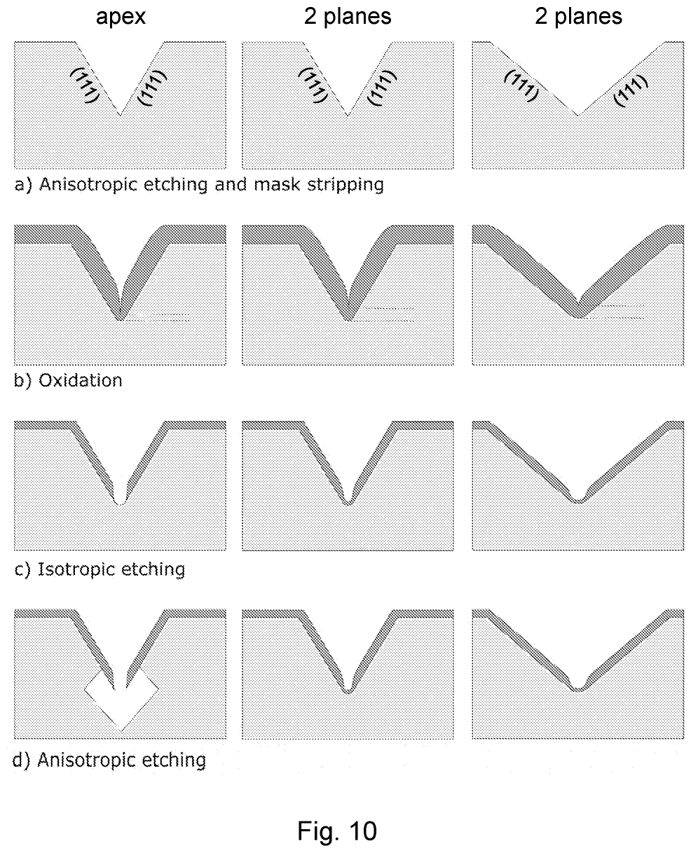

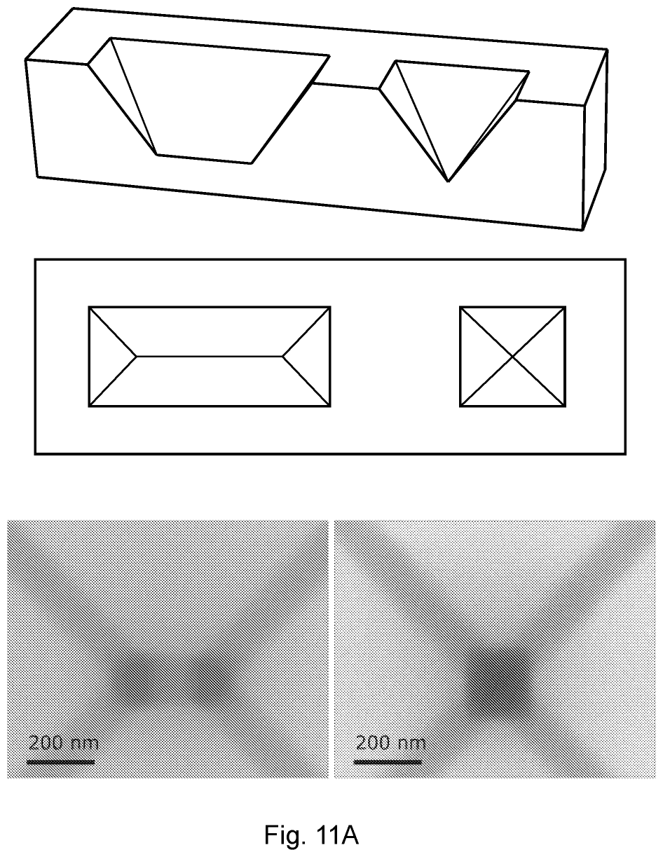

The degree of sharpening of the thermal oxide layer in concave corners depends on the amount of intersecting (111)-planes: the higher the number of intersecting planes, the thinner the grown oxide layer. Thus, in ribbons—i.e. two intersecting (111)-planes—less oxide sharpening occurs compared to an intersection of three or four (111)-planes (i.e. apices) (FIG. 10). These aspects yield the possibility to solely remove the SiO₂ from apices by means of timed isotropic etching in 1% HF, while oxide remains in ribbons and on planes. This is illustrated in FIG. 10.

The procedure to self-form the 3D-fractal now becomes very simple: after thermal oxidation and timed-HF etching, at each apex, the underlying Si can be selectively etched (anisotropic etching in TMAH), resulting in the formation of a next level octahedral structures at all apices simultaneously. Repetition of this simple sequence of anisotropic Si-etching/thermal oxidation at 1100° C./isotropic SiO₂-etching results in multilevel 3D-fractal structures.

### Experimental Results And Discussion

To illustrate the selective opening of apices, we etched an inverted pyramid in (100)-Si using KOH (25 wt.%, 70° C.), with a slightly rectangular (FIG. 11, left), and square (FIG. 11, right) footprint. These structures were subsequently oxidized (dry, 1100° C. for 95 min), resulting in a SiO2 thickness of 160 nm and 155 nm on (111) and (100) oriented surfaces, respectively. FIG. 11 shows SEM images (top view) after 19 min+30 sec etching in 1% HF (etch rate 4.4±0.1 nm/min) and 5 min of TMAH etching (25 wt %, 70° C.) to make a possible opening more visible in the SEM. The remaining oxide thickness on (111) surfaces is 74 nm.

A first indication of the time window (Δt) available between opening of only the apices vs. opening of the ribbons and apices is given in FIG. 11B, for a starting oxide thickness of 88 nm and 160 nm, respectively (on (111) surfaces). For each measurement point in the graphs, the samples were taken from the 1% HF solution, etched in TMAH and then inspected by SEM. This sequence was repeated and the opening of apices or ribbons as detected is indicated in the graphs. Note that the indicated time window has a considerable error margin due to the limited number of measurement points.

Starting point for the realization of 3D fractal structures in an inverted pyramid etched in (100)-Si with KOH, with a square footprint of 5 μm. After growing a thermal oxide layer with a known thickness (ca. 160 nm, 1 h 35 min at 1100° C.), a time window exists for which only the apices are free of oxide. For the engineering of 3D fractal structures solely based on oxide corner-lithography, an etch-time of 20 min 30 sec in 1% HF is applied. Post to this HF-step, through the apex, silicon can be etched anisotropically in TMAH (25 wt. %, 70° C.), yielding a new octahedron that is bound by the slow etching (111) Si-planes. For each fabrication level of a fractal structure, the oxidation and isotropic etch time are constant, however, the time-length of the TMAH etch step is halved for each new level (starting with an etch time of 145 min at level zero). Upon a 3 times repetition of this sequence—TMAH-etching, 1100° C.-oxidation and SiO2-etching—followed by a final thermal oxidation run, anodic bonding with a Mempax glass wafer at 400° C., and removal of the bulk-Si, freestanding three-generation silicon oxide fractal sheets can be fabricated. Note that depending on the final step, apices can remain closed or be opened.

### REFERENCES

[1] E. J. W Berenschot, H. V. Jansen, and N. R. Tas, “Fabrication of 3D fractal structures using nanoscale anistropic etching of single crystalline silicon,” J. Micromech. Microeng., vol. 23, pp. 055024 (10 pp.), 2013.
[2] N. Burouni et al., “Wafer-scale fabrication of nanoapertures using corner lithography,” Nanotechnology, vol. 24, pp. 285303 (10 pp.), 2013.
[3] E. J. W. Berenschot et al., “3D nanofabrication of fluidic components by corner lithography”, Small, vol. 8 (24), pp. 3823-3831, 2012. [4] R. B. Marcus and T. T. Sheng, “The oxidation of shaped silicon surfaces,” J. Electrochem. Soc., vol. 129 (6), pp. 1278-1282, 1982
[5] P. N. Minh, T. Ono, and M. Esashi, “Nonuniform silicon oxidation and application for the fabrication of aperture for near-field scanning optical microscopy,” Appl. Phys. Lett., vol. 75 (26), pp. 4076-4078, 1999.
[6] C. Mihalcea, A. Vollkopf, and E. Oesterschulze, “Reproducible large area microfabrication of sub-100 nm apertures of hollow tips,” J. Electrochem. Soc., vol. 147 (5), pp. 1970-1972, 2000.
[7] B. E. Deal, A. S. Grove, “General relationship for the thermal oxidation of silicon,” J. Appl. Phys., vol. 36, p. 3770, 1965.
[8] A. Vollkopf, O. Rudow, M. Müller-Wiegand, G. Georgiev, and E. Oesterschulze, “Influence of the oxidation temperature on the fabrication process of silicon dioxide aperture tips,” Appl. Phys. A, vol. 76, pp. 923- 926, 2003.

1. Method of producing a cell culture template with at least one three-dimensional structure having a surface maintaining a cell culture, the method comprising the following steps:
step 1: providing a monocrystalline substrate;
step 2: subtracting at least one geometrical feature from the monocrystalline substrate to produce a geometrical cavity in the monocrystalline substrate that renders as the initiation for a three-dimensional structure;
step 3: the growth and/or deposition of a base three-dimensional structure material on the surface of the geometrical features in the substrate to form the three-dimensional structure;
step 4: bonding of the at least one three-dimensional structure to a surface of a support base; and
step 5: removal of the bulk-monocrystalline substrate around the at least one three-dimensional structure;
wherein after removal of the bulk-monocrystalline substrate the surface of the at least one three-dimensional structure is provided with cells under growth permitting conditions to produce the cell culture template.

2. Method for producing a cell culture template comprising at least one three-dimensional structure according to claim 1, wherein the base three-dimensional structure material is silicon nitride or silicon oxide and the cells are provided to the at least one three-dimensional structure at the surface comprising the base three-dimensional material, preferably wherein the base three-dimensional structure material is silicon dioxide, more preferably amorphous silicon dioxide.

3. Method for producing a cell culture template comprising at least one three-dimensional structure according to claim 1 or 2, wherein the at least one three-dimensional structure is a fractal structure, preferably produced by means of micro- and nanofabrication.

4. Method for producing a cell culture template comprising at least one three-dimensional structure according to any one of the preceding claims wherein the monocrystalline substrate is a monocrystalline silicon substrate

5. Method for producing a cell culture template comprising at least one three-dimensional structure according to any one of the preceding claims wherein in step 2 one or more apices are formed.

6. Method for producing a cell culture template comprising at least one three-dimensional structure according to any one of the preceding claims wherein the geometrical cavity is an octahedral cavity or part of an octahedral cavity,

7. Method for producing a cell culture template comprising at least one three-dimensional structure according to any one of the preceding claims wherein the support base is borosilicate glass.

8. Method for producing a cell culture template comprising at least one three-dimensional structure according to any one of the preceding claims, wherein the method further comprises the following steps:
step 6: treating the monocrystalline substrate to form a protective layer which is compatible with the next steps;
step 7: create one or more apertures in the protective layer, preferably an aperture at each of the one or more apices, which is compatible with the following steps;
step 8: subtracting at least one geometrical feature, preferably an octahedron or part of an octahedron, in the monocrystalline substrate through the one or more apertures; followed by stripping the protective layer;
wherein steps 6-8 are performed between step 2 and step 3 of the method of claim 1, optionally repeating steps 6-8 one or more times to create the at least one three-dimensional structure with a higher level of complexity,
preferably wherein steps 6-8 of the method are repeated 2-10 times, preferably 2-5 times to produce three-dimensional structures with higher complexity.

9. Method for producing a cell culture template comprising at least one three-dimensional structure according to claim 8, wherein the protective layer is a base three-dimensional structure material, preferably silicon oxide or silicon nitride, more preferably silicon dioxide.

10. Method for producing a cell culture template comprising at least one three-dimensional structure according to any one of the preceding claims, further comprise
a step 9: providing the at least one three-dimensional structure with an inorganic layer, whereby the inorganic layer is in contact with the base three-dimensional material, whereby said step 9 is performed after step 5 and prior to providing the at least one three-dimensional structure with cells under growth permitting conditions to produce the cell culture template and whereby said cells are provided to the surface of the at least on three-dimensional structure comprising the inorganic layer.

11. Method for producing a cell culture template comprising at least one three-dimensional structure according to any one of the preceding claims, wherein the cavity formed in the monocrystalline substrate of step 2 is accessible from outside the substrate through an opening provided in the substrate by a pre-subtracting directional step,
preferably the opening in the substrate having a relatively large width compared to an average width of the cavity,
more preferably, the opening forming a widest part of the cavity formed in the substrate.

12. Method for producing a cell culture template comprising at least one three-dimensional structure according to any one of the preceding claims, wherein the subtracting is performed by means of anisotropic etching.

13. Method for producing a cell culture template comprising at least one three-dimensional structure according to any one of the preceding claims, wherein the provided monocrystalline substrate is silicon, whereby thermal oxidation results in a layer of silicon oxide, preferably amorphous silicon dioxide, whereby in step 3 a layer of silicon dioxide is deposited and whereby in step 5 the bulk-silicon around the formed three-dimensional structure is removed.

14. Method for producing a cell culture template comprising at least one three-dimensional structure according to any one of the preceding claims,
whereby step 7 is left out at the last round of preparation to produce three-dimensional structures having closed apices.

15. Method for producing a cell culture template comprising at least one three-dimensional structure according to any one of the preceding claims,
wherein the three-dimensional structure comprises a surface defining a regular pattern of protrusions;
the protrusions are built up from octahedral structures; and
the octahedral structures are becoming narrower to the outside of the three-dimensional structure.

16. Method for producing a cell culture template comprising at least one three-dimensional structure according to any one of the preceding claims, wherein the three-dimensional structure has any of the following topographies:
a pyramid (G0),
a pyramid with on the apex an octahedral (G1),
a pyramid with on the apex an octahedral and on each apex of the octahedral a second level of octahedral structures (G2),
a pyramid with on the apex an octahedral and on each apex of the octahedral a second level of octahedral structures and on each apex of the second level a third level of octahedral structures (G3), or
a pyramid with on the apex an octahedral and on each apex of the octahedral a second level of octahedral structures and on each apex of the second level a third level of octahedral structures and on each apex of the third level a fourth level of octahedral structures (G4),
a pyramid with on the apex an octahedral and on each apex of the octahedral a second level of octahedral structures and on each apex of the second level a third level of octahedral structures and on each apex of the third level a fourth level of octahedral structures (G4), on each apex of the n−1th level a nth level of octahedral structures (Gn) n being 5-10.

17. Method for producing a cell culture template comprising at least one three-dimensional structure according to any one of the preceding claims, whereby the three-dimensional structure is sterilized before growing cells, preferably the three-dimensional structure is sterilized by any one of UV, chemical means and high temperature treatment.

18. Method for producing a cell culture template comprising at least one three-dimensional structure according to any one of the preceding claims, wherein the at least one three-dimensional structure comprises multiple three-dimensional structures and wherein the multiple three-dimensional structures are placed on the surface of the support base in a lattice configuration, preferably a square or hexagonal lattice configuration.

19. Method for producing a cell culture template according to claim 18, wherein the bulk-monocrystalline substrate is partially etched away with remaining substrate at least partially covering at least one of the multiple three-dimensional structures.

20. Method for producing a cell culture template according to claim 19, wherein the bulk monocrystalline substrate is partially etched away to create multiple compartments with one or more three-dimensional structures exposed.

21. Method for producing a cell culture template comprising at least one three-dimensional structure according to any one of the preceding claims, wherein the cells are in the form of a tissue or organoid.

22. Method for producing a cell culture template comprising at least one three-dimensional structure according to any one of the preceding claims, wherein the cell culture template further comprises at least one insulator, preferably the insulator is a three-dimensional structure of amorphous silicon dioxide.

23. Method for producing a cell culture template comprising at least one three-dimensional structure according to any one of the preceding claims, wherein the cell culture template further comprises at least one metal portion, preferably the metal portion is embedded or patterned within the three-dimensional structure.

24. Method for producing a cell culture template comprising at least one three-dimensional structure according to claim 22 or 23, wherein the three-dimensional structures are used for external stimulation of the culture.

25. Method for producing a cell culture template comprising at least one three-dimensional structure according to any one of claims 22-24, wherein electrodes are used for cell stimulation, preferably wherein at least part of the three-dimensional structures function as electrodes.

26. Method for producing a cell culture template comprising at least one three-dimensional structure according to any one of the preceding claims, wherein the apices are open and the solutions can be supplied through these apices in the cells culture.

27. Cell culture template for growing and maintaining a cell culture, in particular a cell culture comprising primary cells, the cell culture template comprising cells seeded on a cell growth surface, for example a surface of an amorphous silicon dioxide, the surface defined by at least one three-dimensional fractal structure carried on a support base, for example a layer of borosilicate glass.

28. Cell culture template according to claim 27, wherein the surface is defined by a multitude of, preferably at least almost identical, three-dimensional fractal structures evenly distributed on the support layer.

29. Cell culture template according to claim 28, wherein some of the three-dimensional fractal structures of the multitude of three-dimensional fractal structures on the support layer are covered by monocrystalline substrate with the other three-dimensional fractal structures of the multitude of three-dimensional fractal structures being exposed, i.e. free of monocrystalline, to form the cell growth surface.

30. Cell culture template according to claim 29, wherein the monocrystalline substrate is arranged to define one or more cell growth compartments having one or more exposed fractals.

31. Cell culture template according to claim 29 or 30, wherein a lid is provided on a side of the cell layer opposite of the cell growth surface on top of and supported by the monocrystalline substrate.

32. A method for culturing cells, comprising providing a cell culture template obtainable by a method according to any one of the preceding claims, and culturing the cells.

33. Method for culturing cells or tissues according to claim 31, wherein the cells are primary cells, preferably primary tumour cells.

34. Method for culturing cells or tissues according to claim 32 or 33, wherein the cells are primary cells, preferably primary tissue cells.

35. Method for culturing cells or tissues according to any one of claims 32-34, wherein the cells are cancer-associated fibroblasts (CAFs).

36. Method for culturing cells or tissues according to any one of claims 32-34, wherein the cells are motile cells, preferably activated fibroblasts, further comprising a 1-step isolation and purification of cells or tissues.

37. Method for culturing cells or tissues according to claim 35, wherein the cells are cancer-associated fibroblasts (CAFs) activated by the material, shape, and/or the pattern of the three-dimensional structures.

38. Method for culturing cells or tissues according to any one of claims 32-34, wherein the cells are stem cells, preferably mesenchymal stem cells, adult stem cells, adipose adult stem cells and/or induced pluripotent stem cells.

39. Method for culturing cells or tissues according to any one of claims 32-38, wherein the cells form a multicellular organoid or tissue.

40. Method for culturing cells or tissues according to any one of claims 32-39, wherein the cells undergo stem cell differentiation initiated by the pyramidal shape and the distance of the three-dimensional structures.

41. Method for culturing cells or tissues according to any one of claims 32-40, wherein the cells are grown and be preserved in non-optimal growth conditions.

42. A cell culture template comprising at least one three-dimensional structure obtainable by a method according to any one of claims 1-26, composed of amorphous silicon dioxide and cells attached to the structure.

43. The cell culture template, according to claim 42, wherein the three-dimensional structure of amorphous silicon dioxide consists of SiO₂.

44. Method for producing a three-dimensional structure for cell culture, preferably the three-dimensional structure is a fractal structure, produced by means of micro- and nanofabrication comprising the following steps:
step 1: providing a monocrystalline substrate, preferably a monocrystalline silicon substrate;
step 2: subtracting at least one geometrical feature from the monocrystalline substrate to produce a geometrical cavity, preferably forming one or more apices, preferably an octahedral cavity or part of an octahedral cavity, in the monocrystalline substrate that renders as the initiation for a three-dimensional structure;
step 3: the growth and/or deposition of the base three-dimensional structure material, preferably a silicon oxide, preferably amorphous silicon dioxide, on the surface of the geometrical features in the substrate to form the three-dimensional structure;
step 4: bonding of the at least one three-dimensional structure to a surface of a support base, preferably borosilicate glass; and
step 5: removal of the bulk-monocrystalline substrate around the at least one three-dimensional structure;
wherein after removal of the bulk-monocrystalline substrate the surface of the at least one three-dimensional structure is provided with cells under growth permitting conditions to produce the cell culture template,
optionally, wherein the method further comprises the following steps:
step 6: treating the monocrystalline substrate to form a protective layer which is compatible with the next steps;
step 7: create one or more apertures in the protective layer, preferably an aperture at each of the one or more apices, which is compatible with the following steps;
step 8: subtracting at least one geometrical feature, preferably an octahedron or part of an octahedron, in the monocrystalline substrate through the one or more apertures; followed by stripping the protective layer;
wherein steps 6-8 are performed between step 2 and step 3, optionally repeating steps 6-8 one or more times to create the at least one three-dimensional structure with a higher level of complexity.
